SEQUENCE LISTING (350 sequences)

SEQ ID NO: 1 - protein, 257 aa, Homo sapiens [sequence omitted]

SEQ ID NO: 2 - protein, 15 aa, Homo sapiens
VGLIAARRTGRLRGT

SEQ ID NO: 3 - protein, 15 aa, Homo sapiens
TGRLRGTKTVQEKEN

SEQ ID NO: 4 - DNA, 967 bp, Homo sapiens
gcggcatgggcagtatccgccgccatcctcttccgtgaggcgcgctgagacccggaccggccctcctgagaggatgccggtgcgggcgcccgcggagagggacccgtcgccatgggccgtgtgatccgtggacagaggaagggcgccgggtctgtgttccgcgcgcacgtgaagcaccgtaaaggcgctgcgcgcctgcgcgccgtggatttcgctgagcggcacggctacatcaagggcatcgtcaaggacatcatccacgacccgggccgcggcgcgcccctcgccaaggtggtcttccgggatccgtatcggtttaagaagcggacggagctgttcattgccgccgagggcattcacacgggccagtttgtgtattgcggcaagaaggcccagctcaacgttggcaatgtgctccctgtgggcaccatgcctgagggtacaatcgtgtgctgcctggaggagaagcctggagaccgtggcaagctggcccgggcatcagggaactatgccaccgttatctcccacaaccctgagaccaagaagacccgtgtgaagctgccctccggctccaagaaggttatctcctcagccaacagagctgtggttggtgtggtggctggaggtggccgaattgacaaacccatcttgaaggctggccgggcgtaccacaaatataaggcaaagaggaactgctggccacgagtacggggtgtggccatgaatcctgtggagcatccttttggaggtggcaaccaccagcacatcggcaagccctccaccatccgcagagatgcccctgctggccgcaaagtgggtctcattgctgcccgccggactggacgtctccggggaaccaagactgtgcaggagaaagagaactagtgctgagggcctcaataaagtttgtgtttatgccaaaaaaaaaaaaaaaaaaaaaaaaaaaaaaaaaaaaaaaaaaaaaaaa

SEQ ID NO: 5 - protein, 9 aa, Homo sapiens
AKVVFRDPY

SEQ ID NO: 6 - protein, 9 aa, Homo sapiens
GIHTGQFVY

SEQ ID NO: 7 - protein, 9 aa, Homo sapiens
KLARASGNY

SEQ ID NO: 8 - protein, 9 aa, Homo sapiens
KAGRAYHKY

SEQ ID NO: 9 - protein, 9 aa, Homo sapiens
PILKAGRAY

SEQ ID NO: 10 - protein, 9 aa, Homo sapiens
KLPSGSKKV

SEQ ID NO: 11 - protein, 9 aa, Homo sapiens
SANRAVVGV

SEQ ID NO: 12 - protein, 9 aa, Homo sapiens
YIKGIVKDI

SEQ ID NO: 13 - protein, 9 aa, Homo sapiens
LNIGNVLPV

SEQ ID NO: 14 - protein, 9 aa, Homo sapiens
GRGAPLAKV

SEQ ID NO: 15 - protein, 9 aa, Homo sapiens
RASGNYATV

SEQ ID NO: 16 - protein, 9 aa, Homo sapiens
VISSANRAV

SEQ ID NO: 17 - protein, 9 aa, Homo sapiens
RIDKPILKA

SEQ ID NO: 18 - protein, 9 aa, Homo sapiens
IAARRTGRL

SEQ ID NO: 19 - protein, 9 aa, Homo sapiens
IAAEGIHTG

SEQ ID NO: 20 - protein, 9 aa, Homo sapiens
KAQLNIGNV

SEQ ID NO: 21 - protein, 9 aa, Homo sapiens
APAGRKVGL

SEQ ID NO: 22 - protein, 9 aa, Homo sapiens
KGAARLRAV

SEQ ID NO: 23 - protein, 9 aa, Homo sapiens
GTMPEGTIV

SEQ ID NO: 24 - protein, 9 aa, Homo sapiens
NCWPRVRGV

SEQ ID NO: 25 - protein, 9 aa, Homo sapiens
GRLRGTKTV

SEQ ID NO: 26 - protein, 9 aa, Homo sapiens
IIHDPGRGA

SEQ ID NO: 27 - protein, 9 aa, Homo sapiens
ELFIAAEGI

SEQ ID NO: 28 - protein, 9 aa, Homo sapiens
YRFKKRTEL

SEQ ID NO: 29 - protein, 9 aa, Homo sapiens
VYCGKKAQL

SEQ ID NO: 30 - protein, 9 aa, Homo sapiens
ANRAVVGVV

SEQ ID NO: 31 - protein, 9 aa, Homo sapiens
RDAPAGRKV

SEQ ID NO: 32 - protein, 9 aa, Homo sapiens
VIRGQRKGA

SEQ ID NO: 33 - protein, 9 aa, Homo sapiens
GQRKGAGSV

SEQ ID NO: 34 - protein, 9 aa, Homo sapiens
AGSVFRAHV

SEQ ID NO: 35 - protein, 9 aa, Homo sapiens
KHRKGAARL

SEQ ID NO: 36 - protein, 9 aa, Homo sapiens
FIAAEGIHT

SEQ ID NO: 37 - protein, 9 aa, Homo sapiens
AQLNIGNVL

SEQ ID NO: 38 - protein, 9 aa, Homo sapiens
QLNIGNVLP

SEQ ID NO: 39 - protein, 9 aa, Homo sapiens
VLPVGTMPE

SEQ ID NO: 40 - protein, 9 aa, Homo sapiens
AVVGVVAGG

SEQ ID NO: 41 - protein, 9 aa, Homo sapiens
GVVAGGGRI

SEQ ID NO: 42 - protein, 9 aa, Homo sapiens
ILKAGRAYH

SEQ ID NO: 43 - protein, 9 aa, Homo sapiens
QHIGKPSTI

SEQ ID NO: 44 - protein, 9 aa, Homo sapiens
GLIAARRTG

SEQ ID NO: 45 - protein, 9 aa, Homo sapiens
RVKLPSGSK

SEQ ID NO: 46 - protein, 9 aa, Homo sapiens
ILKAGRAYH

SEQ ID NO: 47 - protein, 9 aa, Homo sapiens
KLARASGNY

SEQ ID NO: 48 - protein, 9 aa, Homo sapiens
RVRGVAMNP

SEQ ID NO: 49 - protein, 9 aa, Homo sapiens
KVGLIAARR

SEQ ID NO: 50 - protein, 9 aa, Homo sapiens
RLRGTKTVQ

SEQ ID NO: 51 - protein, 9 aa, Homo sapiens
HGYIKGIVK

SEQ ID NO: 52 - protein, 9 aa, Homo sapiens
VISHNPETK

SEQ ID NO: 53 - protein, 9 aa, Homo sapiens
RTGRLRGTK

SEQ ID NO: 54 - protein, 9 aa, Homo sapiens
RVIRGQRKG

SEQ ID NO: 55 - protein, 9 aa, Homo sapiens
SVFRAHVKH

SEQ ID NO: 56 - protein, 9 aa, Homo sapiens
RLRAVDFAE

SEQ ID NO: 57 - protein, 9 aa, Homo sapiens
NVLPVGTMP

SEQ ID NO: 58 - protein, 9 aa, Homo sapiens
VKLPSGSKK

SEQ ID NO: 59 - protein, 9 aa, Homo sapiens
AARLRAVDF

SEQ ID NO: 60 - protein, 9 aa, Homo sapiens
PGRGAPLAK

SEQ ID NO: 61 - protein, 9 aa, Homo sapiens
GIHTGQFVY

SEQ ID NO: 62 - protein, 9 aa, Homo sapiens
PILKAGRAY

SEQ ID NO: 63 - protein, 9 aa, Homo sapiens
RAYHKYKAK

SEQ ID NO: 64 - protein, 9 aa, Homo sapiens
GVAMNPVEH

SEQ ID NO: 65 - protein, 9 aa, Homo sapiens
RRDAPAGRK

SEQ ID NO: 66 - protein, 9 aa, Homo sapiens
GLIAARRTG

SEQ ID NO: 67 - protein, 9 aa, Homo sapiens
QRKGAGSVF

SEQ ID NO: 68 - protein, 9 aa, Homo sapiens
GSVFRAHVK

SEQ ID NO: 69 - protein, 9 aa, Homo sapiens
KVVFRDPYR

SEQ ID NO: 70 - protein, 9 aa, Homo sapiens
QLNIGNVLP

SEQ ID NO: 71 - protein, 9 aa, Homo sapiens
KVISSANRA

SEQ ID NO: 72 - protein, 9 aa, Homo sapiens
AVVGVVAGG

SEQ ID NO: 73 - protein, 9 aa, Homo sapiens
LIAARRTGR

SEQ ID NO: 74 - protein, 9 aa, Homo sapiens
VVFRDPYRF

SEQ ID NO: 75 - protein, 9 aa, Homo sapiens
TIVCCLEEK

SEQ ID NO: 76 - protein, 9 aa, Homo sapiens
CLEEKPGDR

SEQ ID NO: 77 - protein, 9 aa, Homo sapiens
GVVAGGGRI

SEQ ID NO: 78 - protein, 9 aa, Homo sapiens
VVAGGGRID

SEQ ID NO: 79 - protein, 9 aa, Homo sapiens
LKAGRAYHK

SEQ ID NO: 80 - protein, 9 aa, Homo sapiens
AGRAYHKYK

SEQ ID NO: 81 - protein, 9 aa, Homo sapiens
TIRRDAPAG

SEQ ID NO: 82 - protein, 9 aa, Homo sapiens
FVYCGKKAQ

SEQ ID NO: 83 - protein, 9 aa, Homo sapiens
GRVIRGQRK

SEQ ID NO: 84 - protein, 9 aa, Homo sapiens
RKGAGSVFR

SEQ ID NO: 85 - protein, 9 aa, Homo sapiens
FAERHGYIK

SEQ ID NO: 86 - protein, 9 aa, Homo sapiens
APLAKVVFR

SEQ ID NO: 87 - protein, 9 aa, Homo sapiens
EEKPGDRGK

SEQ ID NO: 88 - protein, 9 aa, Homo sapiens
SSANRAVVG

SEQ ID NO: 89 - protein, 9 aa, Homo sapiens
GRIDKPILK

SEQ ID NO: 90 - protein, 9 aa, Homo sapiens
QHIGKPSTI

SEQ ID NO: 91 - protein, 9 aa, Homo sapiens
AARRTGRLR

SEQ ID NO: 92 - protein, 9 aa, Homo sapiens
HVKHRKGAA

SEQ ID NO: 93 - protein, 9 aa, Homo sapiens
VKHRKGAAR

SEQ ID NO: 94 - protein, 9 aa, Homo sapiens
RGAPLAKVV

SEQ ID NO: 95 - protein, 9 aa, Homo sapiens
PETKKTRVK

SEQ ID NO: 96 - protein, 9 aa, Homo sapiens
KLPSGSKKV

SEQ ID NO: 97 - protein, 9 aa, Homo sapiens
VVGVVAGGG

SEQ ID NO: 98 - protein, 9 aa, Homo sapiens
VAGGGRIDK

SEQ ID NO: 99 - protein, 9 aa, Homo sapiens
RIDKPILKA

SEQ ID NO: 100 - protein, 9 aa, Homo sapiens
VYCGKKAQL

SEQ ID NO: 101 - protein, 9 aa, Homo sapiens
DFAERHGYI

SEQ ID NO: 102 - protein, 9 aa, Homo sapiens
RFKKRTELF

SEQ ID NO: 103 - protein, 9 aa, Homo sapiens
GYIKGIVKD

SEQ ID NO: 104 - protein, 9 aa, Homo sapiens
APAGRKVGL

SEQ ID NO: 105 - protein, 9 aa, Homo sapiens
WPRVRGVAM

SEQ ID NO: 106 - protein, 9 aa, Homo sapiens
LPSGSKKVI

SEQ ID NO: 107 - protein, 9 aa, Homo sapiens
KPSTIRRDA

SEQ ID NO: 108 - protein, 9 aa, Homo sapiens
KPGDRGKLA

SEQ ID NO: 109 - protein, 9 aa, Homo sapiens
DPGRGAPLA

SEQ ID NO: 110 - protein, 9 aa, Homo sapiens
APLAKVVFR

SEQ ID NO: 111 - protein, 9 aa, Homo sapiens
NPETKKTRV

SEQ ID NO: 112 - protein, 9 aa, Homo sapiens
KPILKAGRA

SEQ ID NO: 113 - protein, 9 aa, Homo sapiens
DPYRFKKRT

SEQ ID NO: 114 - protein, 9 aa, Homo sapiens
KHRKGAARL

SEQ ID NO: 115 - protein, 9 aa, Homo sapiens
HDPGRGAPL

SEQ ID NO: 116 - protein, 9 aa, Homo sapiens
ETKKTRVKL

SEQ ID NO: 117 - protein, 9 aa, Homo sapiens
AEGIHTGQF

SEQ ID NO: 118 - protein, 9 aa, Homo sapiens
PEGTIVCCL

SEQ ID NO: 119 - protein, 9 aa, Homo sapiens
AQLNIGNVL

SEQ ID NO: 120 - protein, 9 aa, Homo sapiens
AARLRAVDF

SEQ ID NO: 121 - protein, 9 aa, Homo sapiens
AMNPVEHPF

SEQ ID NO: 122 - protein, 9 aa, Homo sapiens
ERHGYIKGI

SEQ ID NO: 123 - protein, 9 aa, Homo sapiens
EEKPGDRGK

SEQ ID NO: 124 - protein, 9 aa, Homo sapiens
EKPGDRGKL

SEQ ID NO: 125 - protein, 9 aa, Homo sapiens
APAGRKVGL

SEQ ID NO: 126 - protein, 9 aa, Homo sapiens
AERHGYIKG

SEQ ID NO: 127 - protein, 9 aa, Homo sapiens
ETKKTRVKL

SEQ ID NO: 128 - protein, 9 aa, Homo sapiens
LPSGSKKVI

SEQ ID NO: 129 - protein, 9 aa, Homo sapiens
NPETKKTRV

SEQ ID NO: 130 - protein, 9 aa, Homo sapiens
PAGRKVGLI

SEQ ID NO: 131 - protein, 9 aa, Homo sapiens
RASGNYATV

SEQ ID NO: 132 - protein, 9 aa, Homo sapiens
SANRAVVGV

SEQ ID NO: 133 - protein, 9 aa, Homo sapiens
DPYRFKKRT

SEQ ID NO: 134 - protein, 9 aa, Homo sapiens
DAPAGRKVG

SEQ ID NO: 135 - protein, 9 aa, Homo sapiens
RGAPLAKVV

SEQ ID NO: 136 - protein, 9 aa, Homo sapiens
KAQLNIGNV

SEQ ID NO: 137 - protein, 9 aa, Homo sapiens
VGTMPEGTI

SEQ ID NO: 138 - protein, 9 aa, Homo sapiens
FGGGNHQHI

SEQ ID NO: 139 - protein, 9 aa, Homo sapiens
IAARRTGRL

SEQ ID NO: 140 - protein, 9 aa, Homo sapiens
CGKKAQLNI

SEQ ID NO: 141 - protein, 9 aa, Homo sapiens
APAGRKVGL

SEQ ID NO: 142 - protein, 9 aa, Homo sapiens
KGAARLRAV

SEQ ID NO: 143 - protein, 9 aa, Homo sapiens
APLAKVVFR

SEQ ID NO: 144 - protein, 9 aa, Homo sapiens
IAAEGIHTG

SEQ ID NO: 145 - protein, 9 aa, Homo sapiens
GGGRIDKPI

SEQ ID NO: 146 - protein, 9 aa, Homo sapiens
DFAERHGYI

SEQ ID NO: 147 - protein, 9 aa, Homo sapiens
YIKGIVKDI

SEQ ID NO: 148 - protein, 9 aa, Homo sapiens
LAKVVFRDP

SEQ ID NO: 149 - protein, 9 aa, Homo sapiens
ASGNYATVI

SEQ ID NO: 150 - protein, 9 aa, Homo sapiens
ANRAVVGVV

SEQ ID NO: 151 - protein, 9 aa, Homo sapiens
RAYHKYKAK

SEQ ID NO: 152 - protein, 9 aa, Homo sapiens
AGSVFRAHV

SEQ ID NO: 153 - protein, 9 aa, Homo sapiens
HGYIKGIVK

SEQ ID NO: 154 - protein, 9 aa, Homo sapiens
DPGRGAPLA

SEQ ID NO: 155 - protein, 9 aa, Homo sapiens
GAPLAKVVF

SEQ ID NO: 156 - protein, 9 aa, Homo sapiens
EGIHTGQFV

SEQ ID NO: 157 - protein, 9 aa, Homo sapiens
MPEGTIVCC

SEQ ID NO: 158 - protein, 9 aa, Homo sapiens
LARASGNYA

SEQ ID NO: 159 - protein, 9 aa, Homo sapiens
QHIGKPSTI

SEQ ID NO: 160 - protein, 15 aa, Homo sapiens
RNCWPRVRGVAMNPV

SEQ ID NO: 161 - protein, 15 aa, Homo sapiens
SGNYATVISHNPETK

SEQ ID NO: 162 - protein, 15 aa, Homo sapiens
VKDIIHDPGRGAPLA

SEQ ID NO: 163 - protein, 15 aa, Homo sapiens
RIDKPILKAGRAYHK

SEQ ID NO: 164 - protein, 15 aa, Homo sapiens
GAPLAKVVFRDPYRF

SEQ ID NO: 165 - protein, 15 aa, Homo sapiens
KRTELFIAAEGIHTG

SEQ ID NO: 166 - protein, 15 aa, Homo sapiens
NRAVVGVVAGGGRID

SEQ ID NO: 167 - protein, 15 aa, Homo sapiens
RKGAARLRAVDFAER

SEQ ID NO: 168 - protein, 15 aa, Homo sapiens
LAKVVFRDPYRFKKR

SEQ ID NO: 169 - protein, 15 aa, Homo sapiens
RGKLARASGNYATVI

SEQ ID NO: 170 - protein, 15 aa, Homo sapiens
SSANRAVVGVVAGGG

SEQ ID NO: 171 - protein, 15 aa, Homo sapiens
RAVVGVVAGGGRIDK

SEQ ID NO: 172 - protein, 15 aa, Homo sapiens
WPRVRGVAMNPVEHP

SEQ ID NO: 173 - protein, 15 aa, Homo sapiens
GRKVGLIAARRTGRL

SEQ ID NO: 174 - protein, 15 aa, Homo sapiens
HGYIKGIVKDIIHDP

SEQ ID NO: 175 - protein, 15 aa, Homo sapiens
RDPYRFKKRTELFIA

SEQ ID NO: 176 - protein, 15 aa, Homo sapiens
QLNIGNVLPVGTMPE

SEQ ID NO: 177 - protein, 15 aa, Homo sapiens
VLPVGTMPEGTIVCC

SEQ ID NO: 178 - protein, 15 aa, Homo sapiens
VGLIAARRTGRLRGT

SEQ ID NO: 179 - protein, 15 aa, Homo sapiens
RHGYIKGIVKDIIHD

SEQ ID NO: 180 - protein, 15 aa, Homo sapiens
PYRFKKRTELFIAAE

SEQ ID NO: 181 - protein, 15 aa, Homo sapiens
IGNVLPVGTMPEGTI

SEQ ID NO: 182 - protein, 15 aa, Homo sapiens
GRAYHKYKAKRNCWP

SEQ ID NO: 183 - protein, 15 aa, Homo sapiens
IRGQRKGAGSVFRAH

SEQ ID NO: 184 - protein, 15 aa, Homo sapiens
FIAAEGIHTGQFVYC

SEQ ID NO: 185 - protein, 15 aa, Homo sapiens
KAQLNIGNVLPVGTM

SEQ ID NO: 186 - protein, 15 aa, Homo sapiens
LPVGTMPEGTIVCCL

SEQ ID NO: 187 - protein, 15 aa, Homo sapiens
GSKKVISSANRAVVG

SEQ ID NO: 188 - protein, 15 aa, Homo sapiens
MGRVIRGQRKGAGSV

SEQ ID NO: 189 - protein, 15 aa, Homo sapiens
IKGIVKDIIHDPGRG

SEQ ID NO: 190 - protein, 15 aa, Homo sapiens
TELFIAAEGIHTGQF

SEQ ID NO: 191 - protein, 15 aa, Homo sapiens
IAAEGIHTGQFVYCG

SEQ ID NO: 192 - protein, 15 aa, Homo sapiens
NIGNVLPVGTMPEGT

SEQ ID NO: 193 - protein, 15 aa, Homo sapiens
KPGDRGKLARASGNY

SEQ ID NO: 194 - protein, 15 aa, Homo sapiens
KTRVKLPSGSKKVIS

SEQ ID NO: 195 - protein, 15 aa, Homo sapiens
VVGVVAGGGRIDKPI

SEQ ID NO: 196 - protein, 15 aa, Homo sapiens
YHKYKAKRNCWPRVR

SEQ ID NO: 197 - protein, 15 aa, Homo sapiens
APAGRKVGLIAARRT

SEQ ID NO: 198 - protein, 15 aa, Homo sapiens
RRTGRLRGTKTVQEK

SEQ ID NO: 199 - protein, 15 aa, Homo sapiens
RVIRGQRKGAGSVFR

SEQ ID NO: 200 - protein, 15 aa, Homo sapiens
AVDFAERHGYIKGIV

SEQ ID NO: 201 - protein, 15 aa, Homo sapiens
HTGQFVYCGKKAQLN

SEQ ID NO: 202 - protein, 15 aa, Homo sapiens
QFVYCGKKAQLNIGN

SEQ ID NO: 203 - protein, 15 aa, Homo sapiens
EGTIVCCLEEKPGDR

SEQ ID NO: 204 - protein, 15 aa, Homo sapiens
GTIVCCLEEKPGDRG

SEQ ID NO: 205 - protein, 15 aa, Homo sapiens
TKKTRVKLPSGSKKV

SEQ ID NO: 206 - protein, 15 aa, Homo sapiens
AVVGVVAGGGRIDKP

SEQ ID NO: 207 - protein, 15 aa, Homo sapiens
MNPVEHPFGGGNHQH

SEQ ID NO: 208 - protein, 15 aa, Homo sapiens
EHPFGGGNHQHIGKP

SEQ ID NO: 209 - protein, 15 aa, Homo sapiens
PSTIRRDAPAGRKVG

SEQ ID NO: 210 - protein, 15 aa, Homo sapiens
TIRRDAPAGRKVGLI

SEQ ID NO: 211 - protein, 15 aa, Homo sapiens
ARRTGRLRGTKTVQE

SEQ ID NO: 212 - protein, 15 aa, Homo sapiens
VIRGQRKGAGSVFRA

SEQ ID NO: 213 - protein, 15 aa, Homo sapiens
RAHVKHRKGAARLRA

SEQ ID NO: 214 - protein, 15 aa, Homo sapiens
AHVKHRKGAARLRAV

SEQ ID NO: 215 - protein, 15 aa, Homo sapiens
KDIIHDPGRGAPLAK

SEQ ID NO: 216 - protein, 15 aa, Homo sapiens
IIHDPGRGAPLAKVV

SEQ ID NO: 217 - protein, 15 aa, Homo sapiens
DPGRGAPLAKVVFRD

SEQ ID NO: 218 - protein, 15 aa, Homo sapiens
GKKAQLNIGNVLPVG

SEQ ID NO: 219 - protein, 15 aa, Homo sapiens
LNIGNVLPVGTMPEG

SEQ ID NO: 220 - protein, 15 aa, Homo sapiens
LEEKPGDRGKLARAS

SEQ ID NO: 221 - protein, 15 aa, Homo sapiens
LARASGNYATVISHN

SEQ ID NO: 222 - protein, 15 aa, Homo sapiens
KKTRVKLPSGSKKVI

SEQ ID NO: 223 - protein, 15 aa, Homo sapiens
TRVKLPSGSKKVISS

SEQ ID NO: 224 - protein, 15 aa, Homo sapiens
RVKLPSGSKKVISSA

SEQ ID NO: 225 - protein, 15 aa, Homo sapiens
SKKVISSANRAVVGV

SEQ ID NO: 226 - protein, 15 aa, Homo sapiens
KPILKAGRAYHKYKA

SEQ ID NO: 227 - protein, 15 aa, Homo sapiens
CWPRVRGVAMNPVEH

SEQ ID NO: 228 - protein, 15 aa, Homo sapiens
VRGVAMNPVEHPFGG

SEQ ID NO: 229 - protein, 15 aa, Homo sapiens
GGGNHQHIGKPSTIR

SEQ ID NO: 230 - protein, 15 aa, Homo sapiens
GNHQHIGKPSTIRRD

SEQ ID NO: 231 - protein, 15 aa, Homo sapiens
AGRKVGLIAARRTGR

SEQ ID NO: 232 - protein, 15 aa, Homo sapiens
GRVIRGQRKGAGSVF

SEQ ID NO: 233 - protein, 15 aa, Homo sapiens
AARLRAVDFAERHGY

SEQ ID NO: 234 - protein, 15 aa, Homo sapiens
DIIHDPGRGAPLAKV

SEQ ID NO: 235 - protein, 15 aa, Homo sapiens
PGRGAPLAKVVFRDP

SEQ ID NO: 236 - protein, 15 aa, Homo sapiens
DPYRFKKRTELFIAA

SEQ ID NO: 237 - protein, 15 aa, Homo sapiens
GQFVYCGKKAQLNIG

SEQ ID NO: 238 - protein, 15 aa, Homo sapiens
CGKKAQLNIGNVLPV

SEQ ID NO: 239 - protein, 15 aa, Homo sapiens
VCCLEEKPGDRGKLA

SEQ ID NO: 240 - protein, 15 aa, Homo sapiens
KKVISSANRAVVGVV

SEQ ID NO: 241 - protein, 15 aa, Homo sapiens
GVAMNPVEHPFGGGN

SEQ ID NO: 242 - protein, 15 aa, Homo sapiens
FGGGNHQHIGKPSTI

SEQ ID NO: 243 - protein, 15 aa, Homo sapiens
HQHIGKPSTIRRDAP

SEQ ID NO: 244 - protein, 15 aa, Homo sapiens
DAPAGRKVGLIAARR

SEQ ID NO: 245 - protein, 15 aa, Homo sapiens
TGRLRGTKTVQEKEN

SEQ ID NO: 246 - protein, 15 aa, Homo sapiens
KGIVKDIIHDPGRGA

SEQ ID NO: 247 - protein, 15 aa, Homo sapiens
KKAQLNIGNVLPVGT

SEQ ID NO: 248 - protein, 15 aa, Homo sapiens
PSGSKKVISSANRAV

SEQ ID NO: 249 - protein, 15 aa, Homo sapiens
KKVISSANRAVVGVV

SEQ ID NO: 250 - protein, 15 aa, Homo sapiens
KVVFRDPYRFKKRTE

SEQ ID NO: 251 - protein, 15 aa, Homo sapiens
TELFIAAEGIHTGQF

SEQ ID NO: 252 - protein, 15 aa, Homo sapiens
SGNYATVISHNPETK

SEQ ID NO: 253 - protein, 15 aa, Homo sapiens
RNCWPRVRGVAMNPV

SEQ ID NO: 254 - protein, 15 aa, Homo sapiens
EHPFGGGNHQHIGKP

SEQ ID NO: 255 - protein, 15 aa, Homo sapiens
MGRVIRGQRKGAGSV

SEQ ID NO: 256 - protein, 15 aa, Homo sapiens
LRAVDFAERHGYIKG

SEQ ID NO: 257 - protein, 15 aa, Homo sapiens
HGYIKGIVKDIIHDP

SEQ ID NO: 258 - protein, 15 aa, Homo sapiens
KGIVKDIIHDPGRGA

SEQ ID NO: 259 - protein, 15 aa, Homo sapiens
VKDIIHDPGRGAPLA

SEQ ID NO: 260 - protein, 15 aa, Homo sapiens
AKVVFRDPYRFKKRT

SEQ ID NO: 261 - protein, 15 aa, Homo sapiens
QLNIGNVLPVGTMPE

SEQ ID NO: 262 - protein, 15 aa, Homo sapiens
IGNVLPVGTMPEGTI

SEQ ID NO: 263 - protein, 15 aa, Homo sapiens
RGKLARASGNYATVI

SEQ ID NO: 264 - protein, 15 aa, Homo sapiens
SKKVISSANRAVVGV

SEQ ID NO: 265 - protein, 15 aa, Homo sapiens
VGVVAGGGRIDKPIL

SEQ ID NO: 266 - protein, 15 aa, Homo sapiens
DKPILKAGRAYHKYK

SEQ ID NO: 267 - protein, 15 aa, Homo sapiens
WPRVRGVAMNPVEHP

SEQ ID NO: 268 - protein, 15 aa, Homo sapiens
GVAMNPVEHPFGGGN

SEQ ID NO: 269 - protein, 15 aa, Homo sapiens
GRKVGLIAARRTGRL

SEQ ID NO: 270 - protein, 15 aa, Homo sapiens
KVGLIAARRTGRLRG

SEQ ID NO: 271 - protein, 15 aa, Homo sapiens
RKGAGSVFRAHVKHR

SEQ ID NO: 272 - protein, 15 aa, Homo sapiens
VKHRKGAARLRAVDF

SEQ ID NO: 273 - protein, 15 aa, Homo sapiens
RAVDFAERHGYIKGI

SEQ ID NO: 274 - protein, 15 aa, Homo sapiens
KKTRVKLPSGSKKVI

SEQ ID NO: 275 - protein, 15 aa, Homo sapiens
PSGSKKVISSANRAV

SEQ ID NO: 276 - protein, 15 aa, Homo sapiens
ILKAGRAYHKYKAKR

SEQ ID NO: 277 - protein, 15 aa, Homo sapiens
RDPYRFKKRTELFIA

SEQ ID NO: 278 - protein, 15 aa, Homo sapiens
GSVFRAHVKHRKGAA

SEQ ID NO: 279 - protein, 15 aa, Homo sapiens
RHGYIKGIVKDIIHD

SEQ ID NO: 280 - protein, 15 aa, Homo sapiens
TGQFVYCGKKAQLNI

SEQ ID NO: 281 - protein, 15 aa, Homo sapiens
GRAYHKYKAKRNCWP

SEQ ID NO: 282 - protein, 15 aa, Homo sapiens
VGLIAARRTGRLRGT

SEQ ID NO: 283 - protein, 15 aa, Homo sapiens
SKKVISSANRAVVGV

SEQ ID NO: 284 - protein, 15 aa, Homo sapiens
PYRFKKRTELFIAAE

SEQ ID NO: 285 - protein, 15 aa, Homo sapiens
VGLIAARRTGRLRGT

SEQ ID NO: 286 - protein, 15 aa, Homo sapiens
TGRLRGTKTVQEKEN

SEQ ID NO: 287 - protein, 15 aa, Homo sapiens
HGYIKGIVKDIIHDP

SEQ ID NO: 288 - protein, 15 aa, Homo sapiens
VLPVGTMPEGTIVCC

SEQ ID NO: 289 - protein, 15 aa, Homo sapiens
TRVKLPSGSKKVISS

SEQ ID NO: 290 - protein, 15 aa, Homo sapiens
KKVISSANRAVVGVV

SEQ ID NO: 291 - protein, 15 aa, Homo sapiens
LRAVDFAERHGYIKG

SEQ ID NO: 292 - protein, 15 aa, Homo sapiens
RTELFIAAEGIHTGQ

SEQ ID NO: 293 - protein, 15 aa, Homo sapiens
FIAAEGIHTGQFVYC

SEQ ID NO: 294 - protein, 15 aa, Homo sapiens
RHGYIKGIVKDIIHD

SEQ ID NO: 295 - protein, 15 aa, Homo sapiens
RDPYRFKKRTELFIA

SEQ ID NO: 296 - protein, 15 aa, Homo sapiens
RGKLARASGNYATVI

SEQ ID NO: 297 - protein, 15 aa, Homo sapiens
SGNYATVISHNPETK

SEQ ID NO: 298 - protein, 15 aa, Homo sapiens
RNCWPRVRGVAMNPV

SEQ ID NO: 299 - protein, 15 aa, Homo sapiens
EHPFGGGNHQHIGKP

SEQ ID NO: 300 - protein, 15 aa, Homo sapiens
TELFIAAEGIHTGQF

SEQ ID NO: 301 - protein, 15 aa, Homo sapiens
GQFVYCGKKAQLNIG

SEQ ID NO: 302 - protein, 15 aa, Homo sapiens
KAQLNIGNVLPVGTM

SEQ ID NO: 303 - protein, 15 aa, Homo sapiens
IGNVLPVGTMPEGTI

SEQ ID NO: 304 - protein, 15 aa, Homo sapiens
GTMPEGTIVCCLEEK

SEQ ID NO: 305 - protein, 15 aa, Homo sapiens
ISHNPETKKTRVKLP

SEQ ID NO: 306 - protein, 15 aa, Homo sapiens
RVKLPSGSKKVISSA

SEQ ID NO: 307 - protein, 15 aa, Homo sapiens
YHKYKAKRNCWPRVR

SEQ ID NO: 308 - protein, 15 aa, Homo sapiens
WPRVRGVAMNPVEHP

SEQ ID NO: 309 - protein, 15 aa, Homo sapiens
HQHIGKPSTIRRDAP

SEQ ID NO: 310 - protein, 15 aa, Homo sapiens
RRTGRLRGTKTVQEK

SEQ ID NO: 311 - protein, 15 aa, Homo sapiens
KGIVKDIIHDPGRGA

SEQ ID NO: 312 - protein, 15 aa, Homo sapiens
GVAMNPVEHPFGGGN

SEQ ID NO: 313 - protein, 15 aa, Homo sapiens
HGYIKGIVKDIIHDP

SEQ ID NO: 314 - protein, 15 aa, Homo sapiens
GSVFRAHVKHRKGAA

SEQ ID NO: 315 - protein, 15 aa, Homo sapiens
SVFRAHVKHRKGAAR

SEQ ID NO: 316 - protein, 15 aa, Homo sapiens
SGNYATVISHNPETK

SEQ ID NO: 317 - protein, 15 aa, Homo sapiens
GAPLAKVVFRDPYRF

SEQ ID NO: 318 - protein, 15 aa, Homo sapiens
MGRVIRGQRKGAGSV

SEQ ID NO: 319 - protein, 15 aa, Homo sapiens
GRVIRGQRKGAGSVF

SEQ ID NO: 320 - protein, 15 aa, Homo sapiens
KDIIHDPGRGAPLAK

SEQ ID NO: 321 - protein, 15 aa, Homo sapiens
PGDRGKLARASGNYA

SEQ ID NO: 322 - protein, 15 aa, Homo sapiens
NRAVVGVVAGGGRID

SEQ ID NO: 323 - protein, 15 aa, Homo sapiens
IGKPSTIRRDAPAGR

SEQ ID NO: 324 - protein, 15 aa, Homo sapiens
KVVFRDPYRFKKRTE

SEQ ID NO: 325 - protein, 15 aa, Homo sapiens
RDPYRFKKRTELFIA

SEQ ID NO: 326 - protein, 15 aa, Homo sapiens
TGQFVYCGKKAQLNI

SEQ ID NO: 327 - protein, 15 aa, Homo sapiens
GTIVCCLEEKPGDRG

SEQ ID NO: 328 - protein, 15 aa, Homo sapiens
EHPFGGGNHQHIGKP

SEQ ID NO: 329 - protein, 15 aa, Homo sapiens
GNYATVISHNPETKK

SEQ ID NO: 330 - protein, 15 aa, Homo sapiens
KRNCWPRVRGVAMNP

SEQ ID NO: 331 - protein, 15 aa, Homo sapiens
VKHRKGAARLRAVDF

SEQ ID NO: 332 - protein, 15 aa, Homo sapiens
PETKKTRVKLPSGSK

SEQ ID NO: 333 - protein, 15 aa, Homo sapiens
YHKYKAKRNCWPRVR

SEQ ID NO: 334 - protein, 15 aa, Homo sapiens
KAKRNCWPRVRGVAM

SEQ ID NO: 335 - protein, 15 aa, Homo sapiens
RNCWPRVRGVAMNPV

SEQ ID NO: 336 - protein, 15 aa, Homo sapiens
LIAARRTGRLRGTKT

SEQ ID NO: 337 - protein, 15 aa, Homo sapiens
VFRAHVKHRKGAARL

SEQ ID NO: 338 - protein, 15 aa, Homo sapiens
PGRGAPLAKVVFRDP

SEQ ID NO: 339 - protein, 15 aa, Homo sapiens
APLAKVVFRDPYRFK

SEQ ID NO: 340 - protein, 15 aa, Homo sapiens
RVKLPSGSKKVISSA

SEQ ID NO: 341 - protein, 15 aa, Homo sapiens
VKLPSGSKKVISSAN

SEQ ID NO: 342 - protein, 15 aa, Homo sapiens
KKVISSANRAVVGVV

SEQ ID NO: 343 - protein, 15 aa, Homo sapiens
SSANRAVVGVVAGGG

SEQ ID NO: 344 - protein, 15 aa, Homo sapiens
VAGGGRIDKPILKAG

SEQ ID NO: 345 - protein, 15 aa, Homo sapiens
LKAGRAYHKYKAKRN

SEQ ID NO: 346 - protein, 15 aa, Homo sapiens
HIGKPSTIRRDAPAG

SEQ ID NO: 347 - protein, 15 aa, Homo sapiens
IRRDAPAGRKVGLIA

SEQ ID NO: 348 - protein, 15 aa, Homo sapiens
GRKVGLIAARRTGRL

SEQ ID NO: 349 - protein, 15 aa, Homo sapiens
RKVGLIAARRTGRLR

SEQ ID NO: 350 - protein, 15 aa, Homo sapiens
RPGLLGASVLGLDDI

(19) United States
(12) Patent Application Publication
(10) Pub. No.: US 2010/0080792 A1
(43) Pub. Date: Apr. 1, 2010

(54) Method for Identifying a MHC Class II-Dependent Tumor-Associated T Helper Cell Antigen

(76) Inventors: Dorothee Herlyn, Wynnewood, PA (US); Rajasekharan Somasundaram, West Chester, PA (US); Rolf K. Swoboda, Upper Darby, PA (US)

Correspondence Address: LICATA & TYRRELL P.C., 66 E. MAIN STREET, MARLTON, NJ, 08053, US

(21) Appl. No.: 11/917,363

(22) PCT Filed: Jun. 15, 2006
(86) PCT No.: PCT/US06/23288

§ 371 Date: Jan. 14, 2008

Related U.S. Application Data
(60) Provisional application No. 60/691,029, filed on Jun. 16, 2005.

(51) Int. Cl. A61K 39/395; C40B 20/00; C07K 14/00; A61K 39/00; C12N 5/00; C07K 16/00; A61P 35/00; A61P 31/00

(52) U.S. Cl. 424/130.1; 506/2; 530/350; 424/184.1; 424/277.1; 435/375; 530/389.1; 530/389.7

(57) ABSTRACT

The present invention is a method for identifying MHC class II-dependent disease-associated antigens. The instant method involves expressing a library of disease-derived proteins in lytic bacteriophage for subsequent presentation by antigen presenting cells to T helper cells. Disease-associated antigens are provided as are the use of such antigens in vaccines for inducing an immune response and preventing or treating disease. Moreover, the present invention provides antibodies, which specifically bind to MHC class II-dependent disease-associated antigens or epitope peptides thereof, and their diagnostic and therapeutic use.

This application claims the benefit of U.S. Provisional Patent Application Ser. No. 60/691,029, filed Jun. 16, 2005, the content of which is incorporated herein by reference in its entirety.

This invention was made in the course of research sponsored by the National Institutes of Health (Grant Nos. CA93372-02, CA60975, CA88193, CA25874, CA10815). The U.S. government may have certain rights in this invention.

### BACKGROUND OF THE INVENTION

CD4⁺ T helper (Th) lymphocytes play a central role in the development of protective immunity against tumors and infectious agents. Adoptively transferred CD4⁺ T helper cells, in the absence of CD8⁺ cytolytic T lymphocytes (CTL), inhibit tumor growth in mice (Baskar, et al. (1995) J. Exp. Med. 181:619-29; Dranoff, et al. (1993) Proc. Natl. Acad. Sci. USA 90:3539-43; Hung, et al. (1998) J. Exp. Med. 188:2357-68; Levitsky, et al. (1994) J. Exp. Med. 179:1215-24). Furthermore, the immunotherapeutic potential of MHC class II-associated, tumor-derived peptides has been demonstrated in experimental animals (Hunt, et al. (1992) Science 256:1817-20; Rudensky, et al. (1991) Nature 353:622-7). In melanoma patients, spontaneous tumor regression is associated with CD4⁺ lymphocyte infiltrates (Clemente, et al. (1996) Cancer 77:1303-10; Fischer, et al. (1999) Cancer Immunol. Immunother. 48:363-70). In allogeneic bone marrow transplant patients, the in vivo persistence of adoptively transferred cytomegalovirus-specific CD8⁺ T cells is dependent on an endogenous CD4⁺ T-cell response (Walter, et al. (1995) N. Engl. J. Med. 333:1038-44).

Knowledge of defined human leukocyte antigen (HLA) class II-dependent T helper cell antigens in infectious disease and tumor systems is lacking. Such antigens have great potential for inducing protective immune responses. A few human CD4⁺ T helper cell lines and clones directed against various tumors have been described (Radrizzani, et al. (1991) Int. J. Cancer 49:823-30; Takahashi, et al. (1995) J. Immunol. 154:772-9; Topalian, et al. (1994) Proc. Natl. Acad. Sci. USA 91:9461-5; Topalian, et al. (1994) Int. J. Cancer 58:69-79; Wang (2001) Trends Immunol. 22:269-76). T helper antigens are usually recognized by major histocompatibility complex (MHC) class II-restricted CD4⁺ T helper cells after processing by antigen-presenting cells (APC) through the exogenous pathway (Schwartz (1985) Annu. Rev. Immunol. 3:237-61). Although expression cloning of MHC class II antigens in E. coli has been successful in bacterial and parasitic antigen systems (Sanderson, et al. (1995) J. Exp. Med. 182:1751-7; Mougneau, et al. (1995) Science 268:563-6), this approach has limitations in its application to the human system because of the great complexity of the human genome (Darnell & Baltimore (1986) In: Molecular and Cellular Biology, eds. Lodish, et al., Scientific American Books, New York, pp. 151-188).

The conventional molecular cloning approach of HLA class II-dependent human melanoma and colon carcinoma antigens is based on fusing cDNA tumor libraries to MHC invariant chain (Ii) fragments with the aim of targeting the fusion proteins to the endosomal and lysosomal compartments (Wang (2001) supra) which is necessary for the proteins to be presented in association with MHC class II molecules. Fused libraries are transfected into 293 cells genetically engineered to express DRα, DRβ, DMA, DMB, and Ii and screened for reactivity with CD4⁺ T cells. Using this or slightly modified approaches, six mutated, individual-specific antigens, namely mutated CDC27 (Wang, et al. (1999) Science 284:1351-4), fusion gene LDLR-FUT (Wang, et al. (1999) J. Exp. Med. 189:1659-68), mutated fibronectin (Wang, et al. (2002) J. Exp. Med. 195:1397-406), mutated NeoPAP (Topalian, et al. (2002) Cancer Res. 62:5505-9), mutated PTPRK (Novellino, et al. (2003) J. Immunol. 170:6363-70), and mutated ARTC1 (Wang, et al. (2005) J. Immunol. 174:2661-70) have been identified in melanoma and colorectal carcinoma patients, as have two shared antigens (among patients with the same tumor type), namely COA-1 and EphA3 (Maccalli, et al. (2003) Cancer Res. 63:6735-43; Chiari, et al. (2000) Cancer Res. 60:4855-63). Thus, only two class II-restricted antigens with immunotherapeutic potential for a larger population of patients emerged from these studies.

Needed is a robust method for identifying tumor-associated T helper cell antigens without prior knowledge of the MHC class II restriction elements for use in vaccines for preventing or treating cancer. The present invention meets this need in the art.

### SUMMARY OF THE INVENTION

The present invention is a method for identifying a MHC class II-dependent disease-associated T helper cell antigen. The method involves the steps of expressing a library of disease-derived proteins in lytic bacteriophage; presenting antigens of the library of disease-derived proteins on the surface of MHC class II-positive antigen presenting cells (APC); contacting the APC with T helper cells and determining T helper cell recognition, wherein the recognition by a T helper cells is indicative of said APC presenting a MHC class II-dependent disease-associated T helper cell antigen.

An MHC class II-dependent disease-associated T helper cell antigen and vaccine containing the same are provided as are methods for inducing an immune response to a MHC class II-dependent disease-associated T helper cell antigen and preventing or treating cancer or infectious disease.

Certain embodiments also embrace antibodies which specifically bind to a MHC class II-dependent disease-associated T helper cell antigen or epitope peptide thereof and their use in methods for preventing or treating cancer or infectious disease.

### BRIEF DESCRIPTION OF THE DRAWINGS

FIG. 1 depicts the Ii-cDNA fusion approach (FIG. 1A) (Wang, et al. (1999) supra) and instant bacteriophage-cDNA fusion approach (FIG. 1B) for identifying tumor-associated T helper cell antigens.

FIG. 2 shows that the recognition of RPL8 peptide #2 by Th35-1A cells is HLA DR7− and peptide concentration-dependent. FIG. 2A, Th35-1A cells were stimulated with peptide (between 3.1 and 50 μM)-pulsed autologous DR7⁺ monocytes in the absence of antibody or presence of either control mouse immunoglobulin (Ig) or anti-HLA class II antibody (both at 10 μg/mL). Th35-1A cells were stimulated with peptide (various concentrations)-pulsed autologous monocytes (FIG. 2B), DR7⁺ allogeneic monocytes (FIG. 2C), or DR7⁻ allogeneic monocytes (FIG. 2D). Proliferation of Th35-1A cells was measured by [³H]-thymidine (TdR) incorporation assay. Values with identical symbols (*,#) differ significantly (p<0.01) from each other (FIG. 2A). * denotes experimental values that differ significantly (p<0.01) from the corresponding control values (FIGS. 2B and 2C).

FIG. 3 shows proliferative lymphocyte responses to RPL8 peptide #2 stimulation in PBMC of DR7⁺ melanoma patients. FIGS. 3A-3C, PBMC from three DR7⁺ melanoma patients were stimulated twice with autologous monocytes pulsed with peptide #2 or control peptide, and proliferation ([³H]-TdR incorporation) in PBMC was determined. PBMC from two DR7⁻ melanoma patients (FIGS. 3D and 3E) and four healthy donors (FIG. 3F, only one shown) did not respond after one peptide stimulation. Due to lack of surviving cells after the first round of peptide stimulation, PBMC of DR7⁻ patients or healthy donors could not be stimulated a second time.

### DETAILED DESCRIPTION OF THE INVENTION

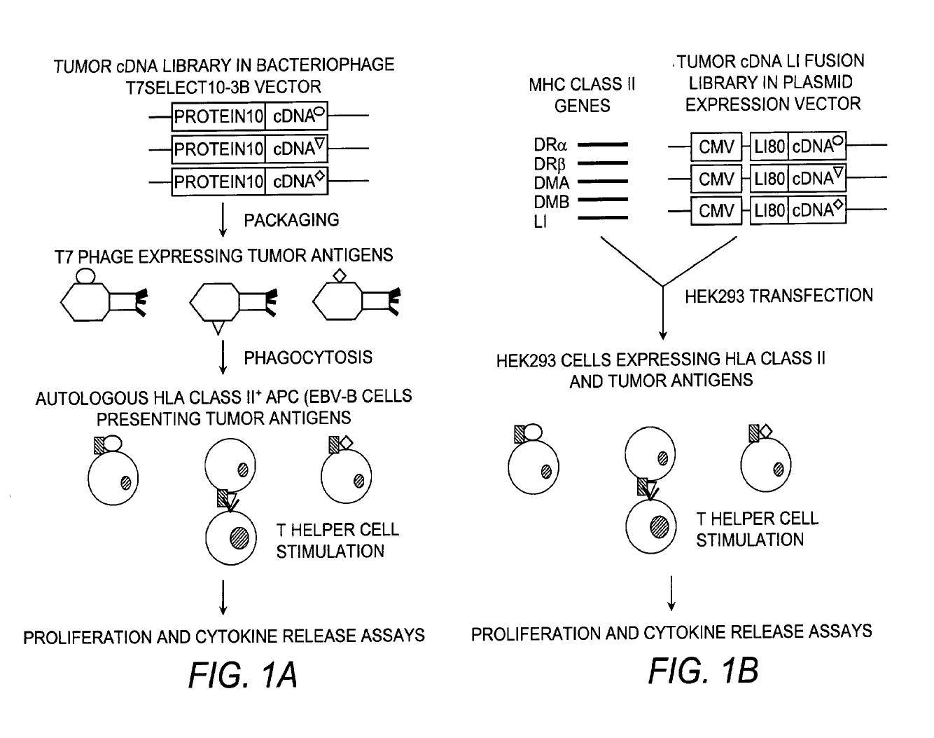

A novel method for identifying disease-associated T helper cell antigens has now been developed. The inventive method involves expressing a library of disease-derived proteins in lytic bacteriophage; presenting the library of disease-derived proteins on the surface of MHC class II-positive antigen presenting cells (APC); contacting the APC with T helper cells and determining T helper cell stimulation, wherein the stimulation of a T helper cell by an APC is indicative of said APC presenting a MHC class II-dependent disease-associated T helper cell antigen (see FIG. 1A). In contrast to conventional methods (FIG. 1B), the instant method provides natural processing of phage-expressed antigen by antigen-presenting cells (APCs) and is not independent on prior knowledge of the MHC restriction molecule used by T helper cells for antigen recognition. Accordingly, relevant disease epitopes are identified which find application in vaccines for the prevention or treatment of diseases such as cancer or infectious disease.

By way of illustration, the instant method was applied to the identification of a melanoma-associated antigen. Th35-1A cells recognize an antigen expressed by melanoma and glioma cells (Somasundaram, et al. (2003) Int. J. Cancer 104:362-8). A cDNA library from WM35 melanoma cells was expressed by T7 phage, APC (EBV-B35 cells) presented phage-library protein to Th35-1A lymphocytes, and the relevant T helper antigen was identified by its capacity to induce proliferation and interferon-γ release in Th35-1A cells. A stimulatory phage clone was identified. The clone had an insert of 185 by and encoded the C-terminal part of ribosomal protein (RP) L8 (Hanes, et al. (1993) Biochem. Biophys. Res. Commun. 197:1223-8; GENBANK Accession No. GI:15082585; SEQ ID NO:1). The cDNA encoded an open reading frame of 58 amino acids.

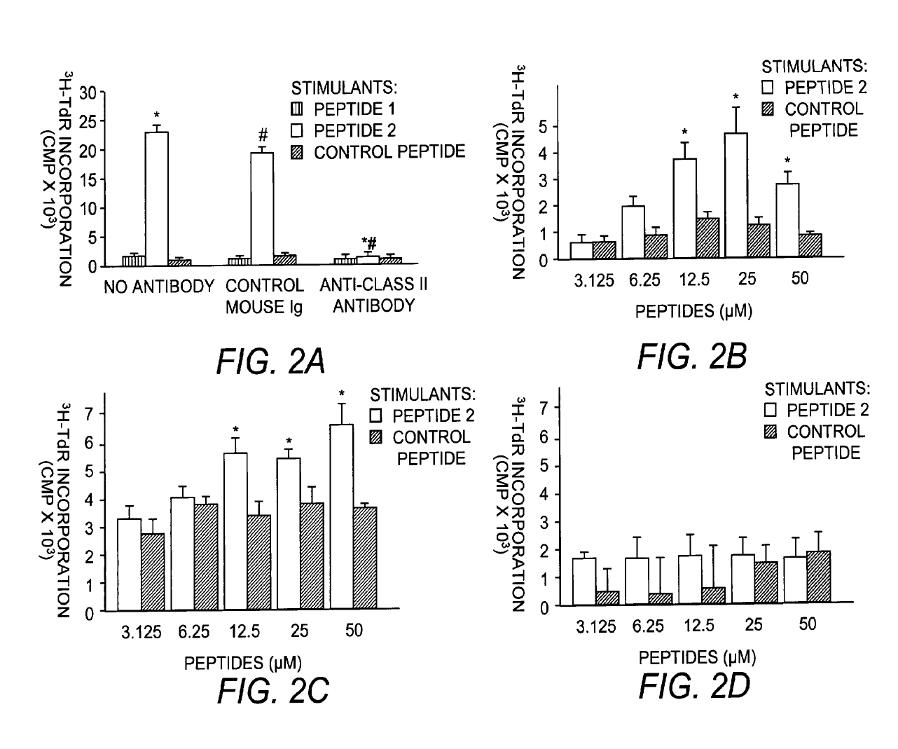

To confirm that RPL8 was recognized by Th35-1A, the peptide epitope recognized by Th35-1A was determined. This epitope was predicted to associate with HLA DR7, as Th35-1A recognizes antigen in association with DR7 (Somasundaram, et al. (2003) supra). The deduced amino acid sequence of the cloned cDNA contains two potential DR7 (DRB1*070101) binding sites (Rammensee, et al. (1999) Immunogenetics 50:213-9). Two overlapping peptides (#1, Val-Gly-Leu-Ile-Ala-Ala-Arg-Arg-Thr-Gly-Arg-Leu-Arg-Gly-Thr; SEQ ID NO:2 and #2, Thr-Gly-Arg-Leu-Arg-Gly-Thr-Lys-Thr-Val-Gln-Glu-Lys-Glu-Asn; SEQ ID NO:3) with high HLA DR7 binding scores (>20) were synthesized and used for stimulation of Th35-1A cells. Peptide #2 (SEQ ID NO:3) was recognized by Th35-1A after presentation by autologous monocytes, and peptide recognition was HLA class II-dependent (FIG. 2A). Th35-1A proliferation was peptide concentration-dependent (FIG. 2B). Allogeneic DR7⁺ monocytes presented peptide #2 to Th35-1A cells (FIG. 2C), whereas DR7⁻ monocytes did not (FIG. 2D).

The data disclosed herein indicate that Th35-1A recognizes RPL8. RPL8 protein (28 kDa) is a component of the 60S subunit of ribosomes and is involved in protein synthesis. It is expressed by all normal cells and ovarian carcinomas (Luo, et al. (2002) Br. J. Cancer 87:339-43). RPL8

RNA is overexpressed in metastatic versus primary carcinomas (Futschik, et al. (2002) Genome Lett. 1:26-34). In light of the ubiquitous expression of RPL8, it was unexpected that some, but not all, tumor cell lysates derived from different patients stimulated proliferation of Th35-1A, although the non-stimulatory tumor cells expressed RPL8 RNA (Table 1) (Somasundaram, et al. (2003) supra).

TABLE 1

| | | Relative RPL8 | Reactivity of |
|---|---|---|---|
| | | RNA | Cell Lysate |
| Cell Name | Cell Type | Abundance¹ | with Th35-1A² |
| FOM 124-1 | Melanocyte | 0.45 | n.d.³ |
| FOM 125-1 | Melanocyte | 0.54 | n.d. |
| WM35 | Melanoma | 1.00 | positive |
| 1205LU | Melanoma | 1.50 | positive |
| WM115 | Melanoma | 0.84 | n.d. |
| WM3450 | Melanoma | 0.93 | n.d. |
| WM3526 | Melanoma | 1.65 | n.d. |
| WM3623 | Melanoma | 1.54 | n.d. |
| WM793 | Melanoma | 0.92 | positive |
| WC020 | Colon Carcinoma | 1.44 | n.d. |
| U87MG | Glioma | 5.00 | positive |
| U373MG | Glioma | 4.50 | positive |
| K562 | Erythroleukemia | 0.36 | negative |
| Daudi | Lymphoma | 1.10 | negative |
| 293 | Human Primary | 1.87 | n.d. |
| | Embryonal Kidney | | |

¹The value of WM35 RNA was set at 1 and the abundance of RNA in the other cells was calculated relative to this value.
²Somasundaram, et al. (2003) supra.
³n.d., not determined.

The nucleotide sequence of full-length RPL8 subsequently cloned from WM35 melanoma cells was 100% identical with the published RPL8 sequence (GENBANK GI:15082585; SEQ ID NO:4). While an antibody to RPL8 was not available to determine RPL8 protein levels in tumors of various tissue origins, RPL8 protein is expressed by melanoma, glioma (as evidenced by recognition of these tumor cells by Th35-1A29) and ovarian carcinoma (Luo, et al. (2002) supra).

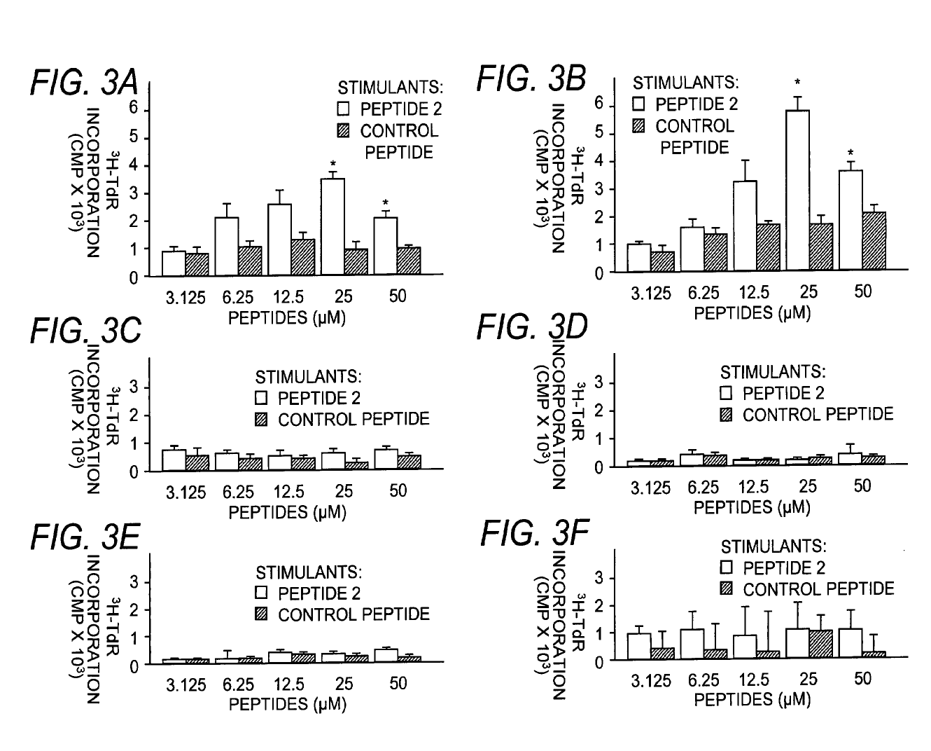

To demonstrate that RPL8 peptide #2 finds application in a vaccine for melanoma patients in addition to patient 35, peripheral blood monocytes from three DR7⁺ melanoma patients were pulsed with the peptide, and proliferation of autologous PBMC following peptide stimulation was determined in [³H]-thymidine incorporation assays. Lymphocytes from two DR7⁻ melanoma patients and four healthy donors served as controls. Lymphocytes from two of the three DR7⁺ melanoma patients (FIGS. 3A-3C) significantly and specifically proliferated to peptide stimulation, whereas neither of the two DR7⁻ melanoma patients (FIGS. 3D and 3E) or four healthy donors (only one donor shown in FIG. 3F) showed lymphoproliferative responses. The results obtained in proliferation assays (FIG. 3) were confirmed in interferon-γ release assays. Thus, the proliferating lymphocytes from the two DR7⁺ patients shown in FIG. 3A and FIG. 3B produced maximally 124.3±1.67 pg and 224.3±4.3 pg per mL of IFN-γ, respectively, whereas the non-proliferating lymphocytes from the two DR7⁻ patients (FIGS. 3D and 3E) and healthy donor (FIG. 3F) produced <12 pg/mL of IFN-γ.

To demonstrate that RPL8 has potential as a vaccine for patients expressing HLA other than DR7, the Rammensee epitope prediction model was used to search for additional putative HLA class II- and class I-binding epitopes on full-length RPL8. Full-length RPL8 contained 27 additional DR7 binding epitopes, and multiple epitopes binding to 3 non-DR7 HLA class II and 7 HLA class I (Tables 2 and 3). Thus, many RPL8 peptides, in addition to peptide #2 and full-length RPL8, are useful in vaccines for cancer patients whose tumors express RPL8, e.g., melanomas, gliomas, and ovarian carcinomas.

TABLE 2

| | | HLA Representation | | |
|---|---|---|---|---|
| | Number of RPL8 | (% of US Population)² | | |
| | Epitopes with a | African | | |
| HLA Type¹ | Binding Score ≧15 | American | Caucasian | Asian |

Class I

| | | | | |
|---|---|---|---|---|
| A0101 | 6 | 5.56 | 15.09 | 1.53 |
| A0201 | 35 | 12.30 | 27.17 | 9.47 |
| A03 | 55 | 9.92 | 12.64 | 0.97 |
| A2402 | 4 | 2.78 | 6.60 | 18.94 |
| B0702 | 13 | 8.17 | 11.13 | 2.51 |
| B4402 | 11 | 1.99 | 11.70 | 0.70 |
| B5101 | 32 | 1.20 | 5.66 | 6.69 |

Class II

| | | | | |
|---|---|---|---|---|
| DRB1*0101 | 89 | 6.82 | 10.22 | 3.46 |
| DRB1*0401 | 34 | 5.70 | 16.75 | 15.46 |
| DRB1*0701 | 28 | 10.13 | 13.28 | 6.92 |
| DRB1*1101 | 39 | 10.61 | 9.31 | 4.73 |

¹Only HLA types expressed by at least 5% of one of the three populations are shown.
²Cao, et al. (2001) Human. Immunol. 62: 1009-1030; Mori, et al. (1997) Transplantation 64: 1017-1027.

TABLE 3

| | | |
|---|---|---|
| | | SEQ |
| | | ID |
| RPL8 Epitope | Score | NO: |

| | | |
|---|---|---|
| HLA-A*01 nonamers | | |
| Val-Asp-Phe-Ala-Glu-Arg-His-Gly-Tyr | 17 | 5 |
| Ala-Lys-Val-Val-Phe-Arg-Asp-Pro-Tyr | 17 | 6 |
| Gly-Ile-His-Thr-Gly-Gln-Phe-Val-Tyr | 17 | 7 |
| Lys-Leu-Ala-Arg-Ala-Ser-Gly-Asn-Tyr | 16 | 8 |
| Lys-Ala-Gly-Arg-Ala-Tyr-His-Lys-Tyr | 16 | 9 |
| Pro-Ile-Leu-Lys-Ala-Gly-Arg-Ala-Tyr | 15 | 10 |
| HLA-A*0201 nonamers | | |
| Lys-Leu-Pro-Ser-Gly-Ser-Lys-Lys-Val | 24 | 11 |
| Ser-Ala-Asn-Arg-Ala-Val-Val-Gly-Val | 24 | 12 |
| Tyr-Ile-Lys-Gly-Ile-Val-Lys-Asp-Ile | 22 | 13 |
| Leu-Asn-Ile-Gly-Asn-Val-Leu-Pro-Val | 21 | 14 |
| Gly-Arg-Gly-Ala-Pro-Leu-Ala-Lys-Val | 20 | 15 |
| Arg-Ala-Ser-Gly-Asn-Tyr-Ala-Thr-Val | 20 | 16 |
| Val-Ile-Ser-Ser-Ala-Asn-Arg-Ala-Val | 20 | 17 |
| Arg-Ile-Asp-Lys-Pro-Ile-Leu-Lys-Ala | 20 | 18 |
| Ile-Ala-Ala-Arg-Arg-Thr-Gly-Arg-Leu | 20 | 19 |
| Ile-Ala-Ala-Glu-Gly-Ile-His-Thr-Gly | 19 | 20 |
| Lys-Ala-Gln-Leu-Asn-Ile-Gly-Asn-Val | 19 | 21 |
| Ala-Pro-Ala-Gly-Arg-Lys-Val-Gly-Leu | 19 | 22 |
| Lys-Gly-Ala-Ala-Arg-Leu-Arg-Ala-Val | 18 | 23 |
| Gly-Thr-Met-Pro-Glu-Gly-Thr-Ile-Val | 18 | 24 |
| Asn-Cys-Trp-Pro-Arg-Val-Arg-Gly-Val | 18 | 25 |
| Gly-Arg-Leu-Arg-Gly-Thr-Lys-Thr-Val | 18 | 26 |
| Ile-Ile-His-Asp-Pro-Gly-Arg-Gly-Ala | 17 | 27 |
| Glu-Leu-Phe-Ile-Ala-Ala-Gln-Gly-Ile | 17 | 28 |
| Tyr-Arg-Phe-Lys-Lys-Arg-Thr-Glu-Leu | 16 | 29 |
| Val-Tyr-Cys-Gly-Lys-Lys-Ala-Gln-Leu | 16 | 30 |
| Ala-Asn-Arg-Ala-Val-Val-Gly-Val-Val | 16 | 31 |
| Arg-Asp-Ala-Pro-Ala-Gly-Arg-Lys-Val | 16 | 32 |
| Val-Ile-Arg-Gly-Gln-Arg-Lys-Gly-Ala | 15 | 33 |
| Gly-Gln-Arg-Lys-Gly-Ala-Gly-Ser-Val | 15 | 34 |
| Ala-Gly-Ser-Val-Phe-Arg-Ala-His-Val | 15 | 35 |
| Lys-His-Arg-Lys-Gly-Ala-Ala-Arg-Leu | 15 | 36 |
| Phe-Ile-Ala-Ala-Glu-Gly-Ile-His-Thr | 15 | 37 |
| Ala-Gln-Leu-Asn-Ile-Gly-Asn-Val-Leu | 15 | 38 |
| Gln-Leu-Asn-Ile-Gly-Asn-Val-Leu-Pro | 15 | 39 |
| Val-Leu-Pro-Val-Gly-Thr-Met-Pro-Glu | 15 | 40 |
| Ala-Val-Val-Gly-Val-Val-Ala-Gly-Gly | 15 | 41 |
| Gly-Val-Val-Ala-Gly-Gly-Gly-Arg-Ile | 15 | 42 |
| Ile-Leu-Lys-Ala-Gly-Arg-Ala-Tyr-His | 15 | 43 |
| Gln-His-Ile-Gly-Lys-Pro-Ser-Thr-Ile | 15 | 44 |
| Gly-Leu-Ile-Ala-Ala-Arg-Arg-Thr-Gly | 15 | 45 |
| HLA-A*03 nonamers | | |
| Arg-Val-Lys-Leu-Pro-Ser-Gly-Ser-Lys | 29 | 46 |
| Ile-Leu-Lys-Ala-Gly-Arg-Ala-Tyr-His | 25 | 47 |
| Lys-Leu-Ala-Arg-Ala-Ser-Gly-Asn-Tyr | 24 | 48 |
| Arg-Val-Arg-Gly-Val-Ala-Met-Asn-Pro | 24 | 49 |
| Lys-Val-Gly-Leu-Ile-Ala-Ala-Arg-Arg | 24 | 50 |
| Arg-Leu-Arg-Gly-Thr-Lys-Thr-Val-Gln | 24 | 51 |
| His-Gly-Tyr-Ile-Lys-Gly-Ile-Val-Lys | 23 | 52 |
| Val-Ile-Ser-His-Asn-Pro-Glu-Thr-Lys | 23 | 53 |
| Arg-Thr-Gly-Arg-Leu-Arg-Gly-Thr-Lys | 23 | 54 |
| Arg-Val-Ile-Arg-Gly-Gln-Arg-Lys-Gly | 22 | 55 |
| Ser-Val-Phe-Arg-Ala-His-Val-Lys-His | 22 | 56 |
| Arg-Leu-Arg-Ala-Val-Asp-Phe-Ala-Glu | 21 | 57 |
| Asn-Val-Leu-Pro-Val-Gly-Thr-Met-Pro | 21 | 58 |
| Val-Lys-Leu-Pro-Ser-Gly-Ser-Lys-Lys | 21 | 59 |
| Ala-Ala-Arg-Leu-Arg-Ala-Val-Asp-Phe | 20 | 60 |
| Pro-Gly-Arg-Gly-Ala-Pro-Leu-Ala-Lys | 20 | 61 |
| Gly-Ile-His-Thr-Gly-Gln-Phe-Val-Tyr | 20 | 62 |
| Pro-Ile-Leu-Lys-Ala-Gly-Arg-Ala-Tyr | 20 | 63 |
| Arg-Ala-Tyr-His-Lys-Tyr-Lys-Ala-Lys | 20 | 64 |
| Gly-Val-Ala-Met-Asn-Pro-Val-Glu-His | 20 | 65 |
| Arg-Arg-Asp-Ala-Pro-Ala-Gly-Arg-Lys | 20 | 66 |
| Gly-Leu-Ile-Ala-Ala-Arg-Arg-Thr-Gly | 20 | 67 |
| Gln-Arg-Lys-Gly-Ala-Gly-Ser-Val-Phe | 19 | 68 |
| Gly-Ser-Val-Phe-Arg-Ala-His-Val-Lys | 19 | 69 |
| Lys-Val-Val-Phe-Arg-Asp-Pro-Tyr-Arg | 19 | 70 |
| Gln-Leu-Asn-Ile-Gly-Asn-Val-Leu-Pro | 19 | 71 |
| Lys-Val-Ile-Ser-Ser-Ala-Asn-Arg-Ala | 19 | 72 |
| Ala-Val-Val-Gly-Val-Val-Ala-Gly-Gly | 19 | 73 |
| Leu-Ile-Ala-Ala-Arg-Arg-Thr-Gly-Arg | 19 | 74 |
| Val-Val-Phe-Arg-Asp-Pro-Tyr-Arg-Phe | 18 | 75 |
| Thr-Ile-Val-Cys-Cys-Leu-Glu-Glu-Lys | 18 | 76 |
| Cys-Leu-Glu-Glu-Lys-Pro-Gly-Asp-Arg | 18 | 77 |
| Gly-Val-Val-Ala-Gly-Gly-Gly-Arg-Ile | 18 | 78 |
| Val-Val-Ala-Gly-Gly-Gly-Arg-Ile-Asp | 18 | 79 |
| Leu-Lys-Ala-Gly-Arg-Ala-Tyr-His-Lys | 18 | 80 |
| Ala-Gly-Arg-Ala-Tyr-His-Lys-Tyr-Lys | 18 | 81 |
| Thr-Ile-Arg-Arg-Asp-Ala-Pro-Ala-Gly | 18 | 82 |
| Phe-Val-Tyr-Cys-Gly-Lys-Lys-Ala-Gln | 17 | 83 |
| Gly-Arg-Val-Ile-Arg-Gly-Gln-Arg-Lys | 16 | 84 |
| Arg-Lys-Gly-Ala-Gly-Ser-Val-Phe-Arg | 16 | 85 |
| Phe-Ala-Glu-Arg-His-Gly-Tyr-Ile-Lys | 16 | 86 |
| Ala-Pro-Leu-Ala-Lys-Val-Val-Phe-Arg | 16 | 87 |
| Glu-Glu-Lys-Pro-Gly-Asp-Arg-Gly-Lys | 16 | 88 |
| Ser-Ser-Ala-Asn-Arg-Ala-Val-Val-Gly | 16 | 89 |
| Gly-Arg-Ile-Asp-Lys-Pro-Ile-Leu-Lys | 16 | 90 |
| Gln-His-Ile-Gly-Lys-Pro-Ser-Thr-Ile | 16 | 91 |
| Ala-Ala-Arg-Arg-Thr-Gly-Arg-Leu-Arg | 16 | 92 |
| His-Val-Lys-His-Arg-Lys-Gly-Ala-Ala | 15 | 93 |
| Val-Lys-His-Arg-Lys-Gly-Ala-Ala-Arg | 15 | 94 |
| Arg-Gly-Ala-Pro-Leu-Ala-Lys-Val-Val | 15 | 95 |
| Pro-Glu-Thr-Lys-Lys-Thr-Arg-Val-Lys | 15 | 96 |
| Lys-Leu-Pro-Ser-Gly-Ser-Lys-Lys-Val | 15 | 97 |
| Val-Val-Gly-Val-Val-Ala-Gly-Gly-Gly | 15 | 98 |
| Val-Ala-Gly-Gly-Gly-Arg-Ile-Asp-Lys | 15 | 99 |
| Arg-Ile-Asp-Lys-Pro-Ile-Leu-Lys-Ala | 15 | 100 |
| HLA-A*2402 nonamers | | |
| Val-Tyr-Cys-Gly-Lys-Lys-Ala-Gln-Leu | 23 | 101 |
| Asp-Phe-Ala-Glu-Arg-His-Gly-Tyr-Ile | 20 | 102 |
| Arg-Phe-Lys-Lys-Arg-Thr-Glu-Leu-Phe | 20 | 103 |
| Gly-Tyr-Ile-Lys-Gly-Ile-Val-Lys-Asp | 16 | 104 |
| HLA-B*0702 nonamers | | |
| Ala-Pro-Ala-Gly-Arg-Lys-Val-Gly-Leu | 27 | 105 |
| Trp-Pro-Arg-Val-Arg-Gly-Val-Ala-Met | 22 | 106 |
| Leu-Pro-Ser-Gly-Ser-Lys-Lys-Val-Ile | 21 | 107 |
| Lys-Pro-Ser-Thr-Ile-Arg-Arg-Asp-Ala | 20 | 108 |
| Lys-Pro-Gly-Asp-Arg-Gly-Lys-Leu-Ala | 19 | 109 |
| Asp-Pro-Gly-Arg-Gly-Ala-Pro-Leu-Ala | 18 | 110 |
| Ala-Pro-Leu-Ala-Lys-Val-Val-Phe-Arg | 17 | 111 |
| Asn-Pro-Glu-Thr-Lys-Lys-Thr-Arg-Val | 17 | 112 |
| Lys-Pro-Ile-Leu-Lys-Ala-Gly-Arg-Ala | 17 | 113 |
| Asp-Pro-Tyr-Arg-Phe-Lys-Lys-Arg-Thr | 16 | 114 |
| Lys-His-Arg-Lys-Gly-Ala-Ala-Arg-Leu | 15 | 115 |
| His-Asp-Pro-Gly-Arg-Gly-Ala-Pro-Leu | 15 | 116 |
| Glu-Thr-Lys-Lys-Thr-Arg-Val-Lys-Leu | 15 | 117 |
| HLA-B*4402 nonamers | | |
| Ala-Glu-Gly-Ile-His-Thr-Gly-Gln-Phe | 25 | 118 |
| Pro-Glu-Gly-Thr-Ile-Val-Cys-Cys-Leu | 23 | 119 |
| Ala-Gln-Leu-Asn-Ile-Gly-Asn-Val-Leu | 18 | 120 |
| Ala-Ala-Arg-Leu-Arg-Ala-Val-Asp-Phe | 17 | 121 |
| Ala-Met-Asn-Pro-Val-Glu-His-Pro-Phe | 17 | 122 |
| Glu-Arg-His-Gly-Tyr-Ile-Lys-Gly-Ile | 16 | 123 |
| Glu-Glu-Lys-Pro-Gly-Asp-Arg-Gly-Lys | 16 | 124 |
| Glu-Lys-Pro-Gly-Asp-Arg-Gly-Lys-Leu | 16 | 125 |
| Ala-Pro-Ala-Gly-Arg-Lys-Val-Gly-Leu | 16 | 126 |
| Ala-Gln-Arg-His-Gly-Tyr-Ile-Lys-Gly | 15 | 127 |
| Glu-Thr-Lys-Lys-Thr-Arg-Val-Lys-Leu | 15 | 128 |
| HLA-B*5101 nonamers | | |
| Leu-Pro-Ser-Gly-Ser-Lys-Lys-Val-Ile | 28 | 129 |
| Asn-Pro-Glu-Thr-Lys-Lys-Thr-Arg-Val | 23 | 130 |
| Pro-Ala-Gly-Arg-Lys-Val-Gly-Leu-Ile | 23 | 131 |
| Arg-Ala-Ser-Gly-Asn-Tyr-Ala-Thr-Val | 21 | 132 |
| Ser-Ala-Asn-Arg-Ala-Val-Val-Gly-Val | 21 | 133 |
| Asp-Pro-Tyr-Arg-Phe-Lys-Lys-Arg-Thr | 20 | 134 |
| Asp-Ala-Pro-Ala-Gly-Arg-Lys-Val-Gly | 20 | 135 |
| Arg-Gly-Ala-Pro-Leu-Ala-Lys-Val-val | 19 | 136 |
| Lys-Ala-Gln-Leu-Asn-Ile-Gly-Asn-Val | 19 | 137 |
| Val-Gly-Thr-Met-Pro-Glu-Gly-Thr-Ile | 19 | 138 |
| Phe-Gly-Gly-Gly-Asn-His-Gln-His-Ile | 19 | 139 |
| Ile-Ala-Ala-Arg-Arg-Thr-Gly-Arg-Leu | 19 | 140 |
| Cys-Gly-Lys-Lys-Ala-Gln-Leu-Asn-Ile | 19 | 141 |
| Ala-Pro-Ala-Gly-Arg-Lys-Val-Gly-Leu | 18 | 142 |
| Lys-Gly-Ala-Ala-Arg-Leu-Arg-Ala-Val | 18 | 143 |
| Ala-Pro-Leu-Ala-Lys-Val-Val-Phe-Arg | 17 | 144 |
| Ile-Ala-Ala-Glu-Gly-Ile-His-Thr-Gly | 17 | 145 |
| Gly-Gly-Gly-Arg-Ile-Asp-Lys-Pro-Ile | 17 | 146 |
| Asp-Phe-Ala-Glu-Arg-His-Gly-Tyr-Ile | 17 | 147 |
| Tyr-Ile-Lys-Gly-Ile-Val-Lys-Asp-Ile | 16 | 148 |
| Leu-Ala-Lys-Va1-Val-Phe-Arg-Asp-Pro | 16 | 149 |
| Ala-Ser-Gly-Asn-Tyr-Ala-Thr-Val-Ile | 16 | 150 |
| Ala-Asn-Arg-Ala-Val-Val-Gly-Val-Val | 16 | 151 |
| Arg-Ala-Tyr-His-Lys-Tyr-Lys-Ala-Lys | 16 | 152 |
| Ala-Gly-Ser-Val-Phe-Arg-Ala-His-Val | 16 | 153 |
| His-Gly-Tyr-Ile-Lys-Gly-Ile-Val-Lys | 15 | 154 |
| Asp-Pro-Gly-Arg-Gly-Ala-Pro-Leu-Ala | 15 | 155 |
| Gly-Ala-Pro-Leu-Ala-Lys-Val-Val-Phe | 15 | 156 |
| Glu-Gly-Ile-His-Thr-Gly-Gln-Phe-Val | 15 | 157 |
| Met-Pro-Glu-Gly-Thr-Ile-Val-Cys-Cys | 15 | 158 |
| Leu-Ala-Arg-Ala-Ser-Gly-Asn-Tyr-Ala | 15 | 159 |
| Gln-His-Ile-Gly-Lys-Pro-Ser-Thr-Ile | 15 | 160 |
| HLA-DRB1*0101 15-mers | | |
| Arg-Asn-Cys-Trp-Pro-Arg-Val-Arg-Gly-Val-Ala-Met-Asn-Pro-Val | 32 | 161 |
| Ser-Gly-Asn-Tyr-Ala-Thr-Val-Ile-Ser-His-Asn-Pro-Glu-Thr-Lys | 30 | 162 |
| Val-Lys-Asp-Ile-Ile-His-Asp-Pro-Gly-Arg-Gly-Ala-Pro-Leu-Ala | 27 | 163 |
| Arg-Ile-Asp-Lys-Pro-Ile-Leu-Lys-Ala-Gly-Arg-Ala-Tyr-His-Lys | 27 | 164 |
| Gly-Ala-Pro-Leu-Ala-Lys-Val-Val-Phe-Arg-Asp-Pro-Tyr-Arg-Phe | 25 | 165 |
| Lys-Arg-Thr-Glu-Leu-Phe-Ile-Ala-Ala-Glu-Gly-Ile-His-Thr-Gly | 25 | 166 |
| Asn-Arg-Ala-Val-Val-Gly-Val-Val-Ala-Gly-Gly-Gly-Arg-Ile-Asp | 25 | 167 |
| Arg-Lys-Gly-Ala-Ala-Arg-Leu-Arg-Ala-Val-Asp-Phe-Ala-Glu-Arg | 24 | 168 |
| Leu-Ala-Lys-Val-Val-Phe-Arg-Asp-Pro-Tyr-Arg-Phe-Lys-Lys-Arg | 24 | 169 |
| Arg-Gly-Lys-Leu-Ala-Arg-Ala-Ser-Gly-Asn-Tyr-Ala-Thr-Val-Ile | 24 | 170 |
| Ser-Ser-Ala-Asn-Arg-Ala-Val-Val-Gly-Val-Val-Ala-Gly-Gly-Gly | 24 | 171 |
| Arg-Ala-Val-Val-Gly-Val-Val-Ala-Gly-Gly-Gly-Arg-Ile-Asp-Lys | 24 | 172 |
| Trp-Pro-Arg-Val-Arg-Gly-Val-Ala-Met-Asn-Pro-Val-Glu-His-Pro | 24 | 173 |
| Gly-Arg-Lys-Val-Gly-Leu-Ile-Ala-Ala-Arg-Arg-Thr-Gly-Arg-Leu | 24 | 174 |
| His-Gly-Tyr-Ile-Lys-Gly-Ile-Val-Lys-Asp-Ile-Ile-His-Asp-Pro | 23 | 175 |
| Arg-Asp-Pro-Tyr-Arg-Phe-Lys-Lys-Arg-Thr-Glu-Leu-Phe-Ile-Ala | 22 | 176 |
| Gln-Leu-Asn-Ile-Gly-Asn-Val-Leu-Pro-Val-Gly-Thr-Met-Pro-Glu | 22 | 177 |
| Val-Leu-Pro-Val-Gly-Thr-Met-Pro-Glu-Gly-Thr-Ile-Val-Cys-Cys | 22 | 178 |
| Val-Gly-Leu-Ile-Ala-Ala-Arg-Arg-Thr-Gly-Arg-Leu-Arg-Gly-Thr | 22 | 179 |
| Arg-His-Gly-Tyr-Ile-Lys-Gly-Ile-Val-Lys-Asp-Ile-Ile-His-Asp | 21 | 180 |
| Pro-Tyr-Arg-Phe-Lys-Lys-Arg-Thr-Glu-Leu-Phe-Ile-Ala-Ala-Glu | 21 | 181 |
| Ile-Gly-Asn-Val-Leu-Pro-Val-Gly-Thr-Met-Pro-Glu-Gly-Thr-Ile | 21 | 182 |
| Gly-Arg-Ala-Tyr-His-Lys-Tyr-Lys-Ala-Lys-Arg-Asn-Cys-Trp-Pro | 21 | 183 |
| Ile-Arg-Gly-Gln-Arg-Lys-Gly-Ala-Gly-Ser-Val-Phe-Arg-Ala-His | 20 | 184 |
| Phe-Ile-Ala-Ala-Glu-Gly-Ile-His-Thr-Gly-Gln-Phe-Val-Tyr-Cys | 20 | 185 |
| Lys-Ala-Gln-Leu-Asn-Ile-Gly-Asn-Val-Leu-Pro-Val-Gly-Thr-Met | 20 | 186 |
| Leu-Pro-Val-Gly-Thr-Met-Pro-Glu-Gly-Thr-Ile-Val-Cys-Cys-Leu | 20 | 187 |
| Gly-Ser-Lys-Lys-Val-Ile-Ser-Ser-Ala-Asn-Arg-Ala-Val-Val-Gly | 20 | 188 |
| Met-Gly-Arg-Val-Ile-Arg-Gly-Gln-Arg-Lys-Gly-Ala-Gly-Ser-Val | 19 | 189 |
| Ile-Lys-Gly-Ile-Val-Lys-Asp-Ile-Ile-His-Asp-Pro-Gly-Arg-Gly | 19 | 190 |
| Thr-Glu-Leu-Phe-Ile-Ala-Ala-Glu-Gly-Ile-His-Thr-Gly-Gln-Phe | 19 | 191 |
| Ile-Ala-Ala-Glu-Gly-Ile-His-Thr-Gly-Gln--Phe-Val-Tyr-Cys-Gly | 19 | 192 |
| Asn-Ile-Gly-Asn-Val-Leu-Pro-Val-Gly-Thr-Met-Pro-Glu-Gly-Thr | 19 | 193 |
| Lys-Pro-Gly-Asp-Arg-Gly-Lys-Leu-Ala-Arg-Ala-Ser-Gly-Asn-Tyr | 19 | 194 |
| Lys-Thr-Arg-Val-Lys-Leu-Pro-Ser-Gly-Ser-Lys-Lys-Val-Ile-Ser | 19 | 195 |
| Val-Val-Gly-Val-Val-Ala-Gly-Gly-Gly-Arg-Ile-Asp-Lys-Pro-Ile | 19 | 196 |
| Tyr-His-Lys-Tyr-Lys-Ala-Lys-Arg-Asn-Cys-Trp-Pro-Arg-Val-Arg | 19 | 197 |
| Ala-Pro-Ala-Gly-Arg-Lys-Val-Gly-Leu-Ile-Ala-Ala-Arg-Arg-Thr | 19 | 198 |
| Arg-Arg-Thr-Gly-Arg-Leu-Arg-Gly-Thr-Lys-Thr-Val-Gln-Glu-Lys | 19 | 199 |
| Arg-Val-Ile-Arg-Gly-Gln-Arg-Lys-Gly-Ala-Gly-Ser-Val-Phe-Arg | 18 | 200 |
| Ala-Val-Asp-Phe-Ala-Glu-Arg-His-Gly-Tyr-Ile-Lys-Gly-Ile-Val | 18 | 201 |
| His-Thr-Gly-Gln-Phe-Val-Tyr-Cys-Gly-Lys-Lys-Ala-Gln-Leu-Asn | 18 | 202 |
| Gln-Phe-Val-Tyr-Cys-Gly-Lys-Lys-Ala-Gln-Leu-Asn-Ile-Gly-Asn | 18 | 203 |
| Glu-Gly-Thr-Ile-Val-Cys-Cys-Leu-Glu-Glu-Lys-Pro-Gly-Asp-Arg | 18 | 204 |
| Gly-Thr-Ile-Val-Cys-Cys-Leu-Glu-Glu-Lys-Pro-Gly-Asp-Arg-Gly | 18 | 205 |
| Thr-Lys-Lys-Thr-Arg-Val-Lys-Leu-Pro-Ser-Gly-Ser-Lys-Lys-Val | 18 | 206 |
| Ala-Val-Val-Gly-Val-Val-Ala-Gly-Gly-Gly-Arg-Ile-Asp-Lys-Pro | 18 | 207 |
| Met-Asn-Pro-Val-Glu-His-Pro-Phe-Gly-Gly-Gly-Asn-His-Gln-His | 18 | 208 |
| Glu-His-Pro-Phe-Gly-Gly-Gly-Asn-His-Gln-His-Ile-Gly-Lys-Pro | 18 | 209 |
| Pro-Ser-Thr-Ile-Arg-Arg-Asp-Ala-Pro-Ala-Gly-Arg-Lys-Val-Gly | 18 | 210 |
| Thr-Ile-Arg-Arg-Asp-Ala-Pro-Ala-Gly-Arg-Lys-Val-Gly-Leu-Ile | 18 | 211 |
| Ala-Arg-Arg-Thr-Gly-Arg-Leu-Arg-Gly-Thr-Lys-Thr-Val-Gln-Glu | 18 | 212 |
| Val-Ile-Arg-Gly-Gln-Arg-Lys-Gly-Ala-Gly-Ser-Val-Phe-Arg-Ala | 17 | 213 |
| Arg-Ala-His-Val-Lys-His-Arg-Lys-Gly-Ala-Ala-Arg-Leu-Arg-Ala | 17 | 214 |
| Ala-His-Val-Lys-His-Arg-Lys-Gly-Ala-Ala-Arg-Leu-Arg-Ala-Val | 17 | 215 |
| Lys-Asp-Ile-Ile-His-Asp-Pro-Gly-Arg-Gly-Ala-Pro-Leu-Ala-Lys | 17 | 216 |
| Ile-Ile-His-Asp-Pro-Gly-Arg-Gly-Ala-Pro-Leu-Ala-Lys-Val-Val | 17 | 217 |
| Asp-Pro-Gly-Arg-Gly-Ala-Pro-Leu-Ala-Lys-Val-Val-Phe-Arg-Asp | 17 | 218 |
| Gly-Lys-Lys-Ala-Gln-Leu-Asn-Ile-Gly-Asn-Val-Leu-Pro-Val-Gly | 17 | 219 |
| Leu-Asn-Ile-Gly-Asn-Val-Leu-Pro-Val-Gly-Thr-Met-Pro-Glu-Gly | 17 | 220 |
| Leu-Glu-Glu-Lys-Pro-Gly-Asp-Arg-Gly-Lys-Leu-Ala-Arg-Ala-Ser | 17 | 221 |
| Leu-Ala-Arg-Ala-Ser-Gly-Asn-Tyr-Ala-Thr-Val-Ile-Ser-His-Asn | 17 | 222 |
| Lys-Lys-Thr-Arg-Val-Lys-Leu-Pro-Ser-Gly-Ser-Lys-Lys-Val-Ile | 17 | 223 |
| Thr-Arg-Val-Lys-Leu-Pro-Ser-Gly-Ser-Lys-Lys-Val-Ile-Ser-Ser | 17 | 224 |
| Arg-Val-Lys-Leu-Pro-Ser-Gly-Ser-Lys-Lys-Val-Ile-Ser-Ser-Ala | 17 | 225 |
| Ser-Lys-Lys-Val-Ile-Ser-Ser-Ala-Asn-Arg-Ala-Val-Val-Gly-Val | 17 | 226 |
| Lys-Pro-Ile-Leu-Lys-Ala-Gly-Arg-Ala-Tyr-His-Lys-Tyr-Lys-Ala | 17 | 227 |
| Cys-Trp-Pro-Arg-Val-Arg-Gly-Val-Ala-Met-Asn-Pro-Val-Glu-His | 17 | 228 |
| Val-Arg-Gly-Val-Ala-Met-Asn-Pro-Val-Glu-His-Pro-Phe-Gly-Gly | 17 | 229 |
| Gly-Gly-Gly-Asn-His-Gln-His-Ile-Gly-Lys-Pro-Ser-Thr-Ile-Arg | 17 | 230 |
| Gly-Asn-His-Gln-His-Ile-Gly-Lys-Pro-Ser-Thr-Ile-Arg-Arg-Asp | 17 | 231 |
| Ala-Gly-Arg-Lys-Val-Gly-Leu-Ile-Ala-Ala-Arg-Arg-Thr-Gly-Arg | 17 | 232 |
| Gly-Arg-Val-Ile-Arg-Gly-Gln-Arg-Lys-Gly-Ala-Gly-Ser-Val-Phe | 16 | 233 |
| Ala-Ala-Arg-Leu-Arg-Ala-Val-Asp-Phe-Ala-Glu-Arg-His-Gly-Tyr | 16 | 234 |
| Asp-Ile-Ile-His-Asp-Pro-Gly-Arg-Gly-Ala-Pro-Leu-Ala-Lys-Val | 16 | 235 |
| Pro-Gly-Arg-Gly-Ala-Pro-Leu-Ala-Lys-Val-Val-Phe-Arg-Asp-Pro | 16 | 236 |
| Asp-Pro-Tyr-Arg-Phe-Lys-Lys-Arg-Thr-Glu-Leu-Phe-Ile-Ala-Ala | 16 | 237 |
| Gly-Gln-Phe-Val-Tyr-Cys-Gly-Lys-Lys-Ala-Gln-Leu-Asn-Ile-Gly | 16 | 238 |
| Cys-Gly-Lys-Lys-Ala-Gln-Leu-Asn-Ile-Gly-Asn-Val-Leu-Pro-Val | 16 | 239 |
| Val-Cys-Cys-Leu-Glu-Glu-Lys-Pro-Gly-Asp-Arg-Gly-Lys-Leu-Ala | 16 | 240 |
| Lys-Lys-Val-Ile-Ser-Ser-Ala-Asn-Arg-Ala-Val-Val-Gly-Val-Val | 16 | 241 |
| Gly-Val-Ala-Met-Asn-Pro-Val-Glu-His-Pro-Phe-Gly-Gly-Gly-Asn | 16 | 242 |
| Phe-Gly-Gly-Gly-Asn-His-Gln-His-Ile-Gly-Lys-Pro-Ser-Thr-Ile | 16 | 243 |
| His-Gln-His-Ile-Gly-Lys-Pro-Ser-Thr-Ile-Arg-Arg-Asp-Ala-Pro | 16 | 244 |
| Asp-Ala-Pro-Ala-Gly-Arg-Lys-Val-Gly-Leu-Ile-Ala-Ala-Arg-Arg | 16 | 245 |
| Thr-Gly-Arg-Leu-Arg-Gly-Thr-Lys-Thr-Val-Gln-Glu-Lys-Glu-Asn | 16 | 246 |
| Lys-Gly-Ile-Val-Lys-Asp-Ile-Ile-His-Asp-Pro-Gly-Arg-Gly-Ala | 15 | 247 |
| Lys-Lys-Ala-Gln-Leu-Asn-Ile-Gly-Asn-Val-Leu-Pro-Val-Gly-Thr | 15 | 248 |
| Pro-Ser-Gly-Ser-Lys-Lys-Val-Ile-Ser-Ser-Ala-Asn-Arg-Ala-Val | 15 | 248 |
| HLA-DRB1*0401 (DR4Dw4) 15-mers | | |
| Lys-Lys-Val-Ile-Ser-Ser-Ala-Asn-Arg-Ala-Val-Val-Gly-Val-Val | 26 | 249 |
| Lys-Val-Val-Phe-Arg-Asp-Pro-Tyr-Arg-Phe-Lys-Lys-Arg-Thr-Glu | 22 | 250 |
| Thr-Glu-Leu-Phe-Ile-Ala-Ala-Glu-Gly-Ile-His-Thr-Gly-Gln-Phe | 22 | 251 |
| Ser-Gly-Asn-Tyr-Ala-Thr-Val-Ile-Ser-His-Asn-Pro-Glu-Thr-Lys | 22 | 252 |
| Arg-Asn-Cys-Trp-Pro-Arg-Val-Arg-Gly-Val-Ala-Met-Asn-Pro-Val | 22 | 253 |
| Gln-His-Pro-Phe-Gly-Gly-Gly-Asn-His-Gln-His-Ile-Gly-Lys-Pro | 22 | 254 |
| Met-Gly-Arg-Val-Ile-Arg-Gly-Gln-Arg-Lys-Gly-Ala-Gly-Ser-Val | 20 | 255 |
| Leu-Arg-Ala-Val-Asp-Phe-Ala-Glu-Arg-His-Gly-Tyr-Ile-Lys-Gly | 20 | 256 |
| His-Gly-Tyr-Ile-Lys-Gly-Ile-Val-Lys-Asp-Ile-Ile-His-Asp-Pro | 20 | 257 |
| Lys-Gly-Ile-Val-Lys-Asp-Ile-Ile-His-Asp-Pro-Gly-Arg-Gly-Ala | 20 | 258 |
| Val-Lys-Asp-Ile-Ile-His-Asp-Pro-Gly-Arg-Gly-Ala-Pro-Leu-Ala | 20 | 259 |
| Ala-Lys-Val-Val-Phe-Arg-Asp-Pro-Tyr-Arg-Phe-Lys-Lys-Arg-Thr | 20 | 260 |
| Gln-Leu-Asn-Ile-Gly-Asn-Val-Leu-Pro-Val-Gly-Thr-Met-Pro-Glu | 20 | 261 |
| Ile-Gly-Asn-Val-Leu-Pro-Val-Gly-Thr-Met-Pro-Glu-Gly-Thr-Ile | 20 | 262 |
| Arg-Gly-Thys-Leu-Ala-Arg-Ala-Ser-Gly-Asn-Tyr-Ala-Thr-Val-Ile | 20 | 263 |
| Ser-Lys-Lys-Val-Ile-Ser-Ser-Ala-Asn-Arg-Ala-Val-Val-Gly-Val | 20 | 264 |
| Val-Gly-Val-Val-Ala-Gly-Gly-Gly-Arg-Ile-Asp-Lys-Pro-Ile-Leu | 20 | 265 |
| Asp-Lys-Pro-Ile-Leu-Lys-Ala-Gly-Arg-Ala-Tyr-His-Lys-Tyr-Lys | 20 | 266 |
| Trp-Pro-Arg-Val-Arg-Gly-Val-Ala-Met-Asn-Pro-Val-Glu-His-Pro | 20 | 267 |
| Gly-Val-Ala-Met-Asn-Pro-Val-Glu-His-Pro-Phe-Gly-Gly-Gly-Asn | 20 | 268 |
| Gly-Arg-Lys-Val-Gly-Leu-11e-Ala-Ala-Arg-Arg-Thr-Gly-Arg-Leu | 20 | 269 |
| Lys-Val-Gly-Leu-Ile-Ala-Ala-Arg-Arg-Thr-Gly-Arg-Leu-Arg-Gly | 20 | 270 |
| Arg-Lys-Gly-Ala-Gly-Ser-Val-Phe-Arg-Ala-His-Val-Lys-His-Arg | 18 | 271 |
| Val-Lys-His-Arg-Lys-Gly-Ala-Ala-Arg-Leu-Arg-Ala-Val-Asp-Phe | 18 | 272 |
| Arg-Ala-Val-Asp-Phe-Ala-Glu-Arg-His-Gly-Tyr-Ile-Lys-Gly-Ile | 18 | 273 |
| Lys-Lys-Thr-Arg-Val-Lys-Leu-Pro-Ser-Gly-Ser-Lys-Lys-Val-Ile | 18 | 274 |
| Pro-Ser-Gly-Ser-Lys-Lys-Val-Ile-Ser-Ser-Ala-Asn-Arg-Ala-Val | 18 | 275 |
| Ile-Leu-Lys-Ala-Gly-Arg-Ala-Tyr-His-Lys-Tyr-Lys-Ala-Lys-Arg | 18 | 276 |
| Arg-Asp-Pro-Tyr-Arg-Phe-Lys-Lys-Arg-Thr-Glu-Leu-Phe-Ile-Ala | 17 | 277 |
| Gly-Ser-Val-Phe-Arg-Ala-His-Val-Lys-His-Arg-Lys-Gly-Ala-Ala | 16 | 278 |
| Arg-His-Gly-Tyr-Ile-Lys-Gly-Ile-Val-Lys-Asp-Ile-Ile-His-Asp | 16 | 279 |
| Thr-Gly-Gln-Phe-Val-Tyr-Cys-Gly-Lys-Lys-Ala-Gln-Leu-Asn-Ile | 16 | 280 |
| Gly-Arg-Ala-Tyr-His-Lys-Tyr-Lys-Ala-Lys-Arg-Asn-Cys-Trp-Pro | 16 | 281 |
| Val-Gly-Leu-Ile-Ala-Ala-Arg-Arg-Thr-Gly-Arg-Leu-Arg-Gly-Thr | 15 | 282 |
| HLA-DRB1*0701 15-mers | | |
| Ser-Lys-Lys-Val-Ile-Ser-Ser-Ala-Asn-Arg-Ala-Val-Val-Gly-Val | 28 | 283 |
| Pro-Tyr-Arg-Phe-Lys-Lys-Arg-Thr-Glu-Leu-Phe-Ile-Ala-Ala-Glu | 24 | 284 |
| Val-Gly-Leu-Ile-Ala-Ala-Arg-Arg-Thr-Gly-Arg-Leu-Arg-Gly-Thr | 24 | 285 |
| Thr-Gly-Arg-Leu-Arg-Gly-Thr-Lys-Thr-Val-Gln-Glu-Lys-Glu-Asn | 24 | 286 |
| His-Gly-Tyr-Ile-Lys-Gly-Ile-Val-Lys-Asp-Ile-Ile-His-Asp-Pro | 22 | 287 |
| Val-Leu-Pro-Val-Gly-Thr-Met-Pro-Glu-Gly-Thr-Ile-Val-Cys-Cys | 22 | 288 |
| Thr-Arg-Val-Lys-Leu-Pro-Ser-Gly-Ser-Lys-Lys-Val-Ile-Ser-Ser | 22 | 289 |
| Lys-Lys-Val-Ile-Ser-Ser-Ala-Asn-Arg-Ala-Val-Val-Gly-Val-Val | 22 | 290 |
| Leu-Arg-Ala-Val-Asp-Phe-Ala-Glu-Arg-His-Gly-Tyr-Ile-Lys-Gly | 20 | 291 |
| Arg-Thr-Glu-Leu-Phe-Ile-Ala-Ala-Glu-Gly-Ile-His-Thr-Gly-Gln | 20 | 292 |
| Phe-Ile-Ala-Ala-Glu-Gly-Ile-His-Thr-Gly-Gln-Phe-Val-Tyr-Cys | 20 | 293 |
| Arg-His-Gly-Tyr-Ile-Lys-Gly-Ile-Val-Lys-Asp-Ile-Ile-His-Asp | 18 | 294 |
| Arg-Asp-Pro-Tyr-Arg-Phe-Lys-Lys-Arg-Thr-Glu-Leu-Phe-Ile-Ala | 18 | 295 |
| Arg-Gly-Lys-Leu-Ala-Arg-Ala-Ser-Gly-Asn-Tyr-Ala-Thr-Val-Ile | 18 | 296 |
| Ser-Gly-Asn-Tyr-Ala-Thr-Val-Ile-Ser-His-Asn-Pro-Glu-Thr-Lys | 18 | 297 |
| Arg-Asn-Cys-Trp-Pro-Arg-Val-Arg-Gly-Val-Ala-Met-Asn-Pro-Val | 18 | 298 |
| Glu-His-Pro-Phe-Gly-Gly-Gly-Asn-His-Gln-His-Ile-Gly-Lys-Pro | 18 | 299 |
| Thr-Glu-Leu-Phe-Ile-Ala-Ala-Glu-Gly-Ile-His-Thr-Gly-Gln-Phe | 16 | 300 |
| Gly-Gln-Phe-Val-Tyr-Cys-Gly-Lys-Lys-Ala-Gln-Leu-Asn-Ile-Gly | 16 | 301 |
| Lys-Ala-Gln-Leu-Asn-Ile-Gly-Asn-Val-Leu-Pro-Val-Gly-Thr-Met | 16 | 302 |
| Ile-Gly-Asn-Val-Leu-Pro-Val-Gly-Thr-Met-Pro-Glu-Gly-Thr-Ile | 16 | 303 |
| Gly-Thr-Met-Pro-Glu-Gly-Thr-Ile-Val-Cys-Cys-Leu-Glu-Glu-Lys | 16 | 304 |
| Ile-Ser-His-Asn-Pro-Glu-Thr-Lys-Lys-Thr-Arg-Val-Lys-Leu-Pro | 16 | 305 |
| Arg-Val-Lys-Leu-Pro-Ser-Gly-Ser-Lys-Lys-Val-Ile-Ser-Ser-Ala | 16 | 306 |
| Tyr-His-Lys-Tyr-Lys-Ala-Lys-Arg-Asn-Cys-Trp-Pro-Arg-Val-Arg | 16 | 307 |
| Trp-Pro-Arg-Val-Arg-Gly-Val-Ala-Met-Asn-Pro-Val-Glu-His-Pro | 16 | 308 |
| His-Gln-His-Ile-Gly-Lys-Pro-Ser-Thr-Ile-Arg-Arg-Asp-Ala-Pro | 16 | 309 |
| Arg-Arg-Thr-Gly-Arg-Leu-Arg-Gly-Thr-Lys-Thr-Val-Gln-Glu-Lys | 16 | 310 |
| HLA-DRB1*1101 15-mers | | |
| Lys-Gly-Ile-Val-Lys-Asp-Ile-Ile-His-Asp-Pro-Gly-Arg-Gly-Ala | 26 | 311 |
| Gly-Val-Ala-Met-Asn-Pro-Val-Glu-His-Pro-Phe-Gly-Gly-Gly-Asn | 26 | 312 |
| His-Gly-Tyr-Ile-Lys-Gly-Ile-Val-Lys-Asp-Ile-Ile-His-Asp-Pro | 23 | 313 |
| Gly-Ser-Val-Phe-Arg-Ala-His-Val-Lys-His-Arg-Lys-Gly-Ala-Ala | 22 | 314 |
| Ser-Val-Phe-Arg-Ala-His-Val-Lys-His-Arg-Lys-Gly-Ala-Ala-Arg | 22 | 315 |
| Ser-Gly-Asn-Tyr-Ala-Thr-Val-Ile-Ser-His-Asn-Pro-Glu-Thr-Lys | 22 | 316 |
| Gly-Ala-Pro-Leu-Ala-Lys-Val-Val-Phe-Arg-Asp-Pro-Tyr-Arg-Phe | 21 | 317 |
| Met-Gly-Arg-Val-Ile-Arg-Gly-Gln-Arg-Lys-Gly-Ala-Gly-Ser-Val | 20 | 318 |
| Gly-Arg-Val-Ile-Arg-Gly-Gln-Arg-Lys-Gly-Ala-Gly-Ser-Val-Phe | 20 | 319 |
| Lys-Asp-Ile-Ile-His-Asp-Pro-Gly-Arg-Gly-Ala-Pro-Leu-Ala-Lys | 20 | 320 |
| Pro-Gly-Asp-Arg-Gly-Lys-Leu-Ala-Arg-Ala-Ser-Gly-Asn-Tyr-Ala | 20 | 321 |
| Asn-Arg-Ala-Val-Val-Gly-Val-Val-Ala-Gly-Gly-Gly-Arg-Ile-Asp | 20 | 322 |
| Ile-Gly-Lys-Pro-Ser-Thr-Ile-Arg-Arg-Asp-Ala-Pro-Ala-Gly-Arg | 20 | 323 |
| Lys-Val-Val-Phe-Arg-Asp-Pro-Tyr-Arg-Phe-Lys-Lys-Arg-Thr-Glu | 19 | 324 |
| Arg-Asp-Pro-Tyr-Arg-Phe-Lys-Lys-Arg-Thr-Glu-Leu-Phe-Ile-Ala | 18 | 325 |
| Thr-Gly-Gln-Phe-Val-Tyr-Cys-Gly-Lys-Lys-Ala-Gln-Leu-Asn-Ile | 18 | 326 |
| Gly-Thr-Ile-Val-Cys-Cys-Leu-Glu-Glu-Lys-Pro-Gly-Asp-Arg-Gly | 18 | 327 |
| Glu-His-Pro-Phe-Gly-Gly-Gly-Asn-His-Gln-His-Ile-Gly-Lys-Pro | 18 | 328 |
| Gly-Asn-Tyr-Ala-Thr-Val-Ile-Ser-His-Asn-Pro-Glu-Thr-Lys-Lys | 17 | 329 |
| Lys-Arg-Asn-Cys-Trp-Pro-Arg-Val-Arg-Gly-Val-Ala-Met-Asn-Pro | 17 | 330 |
| Val-Lys-His-Arg-Lys-Gly-Ala-Ala-Arg-Leu-Arg-Ala-Val-Asp-Phe | 16 | 331 |
| Pro-Glu-Thr-Lys-Lys-Thr-Arg-Val-Lys-Leu-Pro-Ser-Gly-Ser-Lys | 16 | 332 |
| Tyr-His-Lys-Tyr-Lys-Ala-Lys-Arg-Asn-Cys-Trp-Pro-Arg-Val-Arg | 16 | 333 |
| Lys-Ala-Lys-Arg-Asn-Cys-Trp-Pro-Arg-Val-Arg-Gly-Val-Ala-Met | 16 | 334 |
| Arg-Asn-Cys-Trp-Pro-Arg-Val-Arg-Gly-Val-Ala-Met-Asn-Pro-Val | 16 | 335 |
| Leu-Ile-Ala-Ala-Arg-Arg-Thr-Gly-Arg-Leu-Arg-Gly-Thr-Lys-Thr | 16 | 336 |
| Val-Phe-Arg-Ala-His-Val-Lys-His-Arg-Lys-Gly-Ala-Ala-Arg-Leu | 15 | 337 |
| Pro-Gly-Arg-Gly-Ala-Pro-Leu-Ala-Lys-Val-Val-Phe-Arg-Asp-Pro | 15 | 338 |
| Ala-Pro-Leu-Ala-Lys-Val-Val-Phe-Arg-Asp-Pro-Tyr-Arg-Phe-Lys | 15 | 339 |
| Arg-Val-Lys-Leu-Pro-Ser-Gly-Ser-Lys-Lys-Val-Ile-Ser-Ser-Ala | 15 | 340 |
| Val-Lys-Leu-Pro-Ser-Gly-Ser-Lys-Lys-Val-Ile-Ser-Ser-Ala-Asn | 15 | 341 |
| Lys-Lys-Val-Ile-Ser-Ser-Ala-Asn-Arg-Ala-Val-Val-Gly-Val-Val | 15 | 342 |
| Ser-Ser-Ala-Asn-Arg-Ala-Val-Val-Gly-Val-Val-Ala-Gly-Gly-Gly | 15 | 343 |
| Val-Ala-Gly-Gly-Gly-Arg-Ile-Asp-Lys-Pro-Ile-Leu-Lys-Ala-Gly | 15 | 344 |
| Leu-Lys-Ala-Gly-Arg-Ala-Tyr-His-Lys-Tyr-Lys-Ala-Lys-Arg-Asn | 15 | 345 |
| His-Ile-Gly-Lys-Pro-Ser-Thr-Ile-Arg-Arg-Asp-Ala-Pro-Ala-Gly | 15 | 346 |
| Ile-Arg-Arg-Asp-Ala-Pro-Ala-Gly-Arg-Lys-Val-Gly-Leu-Ile-Ala | 15 | 347 |
| Gly-Arg-Lys-Val-Gly-Leu-Ile-Ala-Ala-Arg-Arg-Thr-Gly-Arg-Leu | 15 | 348 |
| Arg-Lys-Val-Gly-Leu-Ile-Ala-Ala-Arg-Arg-Thr-Gly-Arg-Leu-Arg | 15 | 349 |

Having demonstrated the identification of a melanoma tumor antigen using the instant method, one of skill in the art can readily appreciate the broad application of the instant screening method for identifying MHC class II-dependent tumor-associated T helper cell antigens for other cancers as well as infectious agents. In contrast to the conventional cDNA library-Ii fusion approach (Wang (2001) supra), the instant method advantageously does not require prior knowledge of the MHC class II restriction element as proteins expressed from tumor cDNA libraries are presented to T helper cells by MHC class II-positive B cells, wherein the antigenic regions of said proteins have been naturally processed by APCs. Further, instead of the lysogenic filamentous phage commonly used in phage display libraries, lytic phage were employed. Advantages for using lytic phage such as T7 include the fact that the cDNA is located at the 3′ end of protein 10B, requiring only one correct reading frame fusion, whereas in filamentous phage the cDNA is located in the middle of pIII, thus requiring two in-frame fusions; and the lytic life cycle of T7 phage avoids negative selection of proteins during protein transport through the bacterial membrane, which is necessary for assembling filamentous phage.

As used in the context of the present invention, Major Histocompatibility Complex (MHC) is a generic designation meant to encompass the histo-compatibility antigen systems described in different species, including the human leukocyte antigens (HLA). In contrast to MHC class I, MHC class II molecules are found on B cells, macrophages and other antigen presenting cells, collectively referred to herein as MHC class II-positive APCs. MHC class II-positive APCs facilitate the elicitation of an immune response to an antigen by presenting the antigen to T helper cells. Such antigens are designated herein as being MHC class II-dependent. MHC class II-dependent antigens of particular interest in the present invention are disease-associated antigens including tumor-associated and infectious agent-associated antigens. In certain embodiments, a disease-associated antigen is a protein or peptide unique to a tumor cell or infectious agent which can elicit an immune response in a subject, including a cellular or humoral immune response.

The instant method finds application in the identification of tumor-associated antigens from cancers including, but not limited to, melanomas, metastases, adenocarcinoma, thymoma, lymphoma, sarcoma, lung cancer, liver cancer, colon cancer, non-Hodgkins lymphoma, Hodgkins lymphoma, leukemias, uterine cancer, breast cancer, prostate cancer, ovarian cancer, cervical cancer, bladder cancer, kidney cancer, pancreatic cancer and others.

Examples of infectious agents for which MHC class II-dependent antigens can be identified include, but are not limited to, viruses such Hepadnaviridae including hepatitis B virus (HBV); Flaviviridae including human hepatitis C virus (HCV), yellow fever virus and dengue viruses; Retroviridae including human immunodeficiency viruses (HIV) and human T lymphotropic viruses (HTLV1 and HTLV2); Herpesviridae including herpes simplex viruses (HSV-1 and HSV-2), Epstein Barr virus (EBV), cytomegalovirus, varicella-zoster virus (VZV), human herpes virus 6 (HHV-6) human herpes virus 8 (HHV-8), and herpes B virus; Papovaviridae including human papilloma viruses; Rhabdoviridae including rabies virus; Paramyxoviridae including respiratory syncytial virus; Reoviridae including rotaviruses; Bunyaviridae including hantaviruses; Filoviridae including Ebola virus; Adenoviridae; Parvoviridae including parvovirus B-19; Arenaviridae including Lassa virus; Orthomyxoviridae including influenza viruses; Poxyiridae including Orf virus and Monkey pox virus; Togaviridae; Coronaviridae including corona viruses; and Picornaviridae.

Non-viral infectious agents include, e.g., pathogenic protozoa such as Pneumocystis carinii, Trypanosoma, Leishmania, Plasmodia, and Toxoplasma gondii; bacteria such as Mycobacteria, and Legioniella; and fungi such as Histoplasma capsulatum and Coccidioides immitis.

MHC class II-dependent disease-associated antigens are identified in accordance with the present invention by expressing a library of disease-derived proteins in lytic bacteriophage for subsequent presentation by antigen presenting cells to T helper cells. The term “library of disease-derived proteins”, when used in the context of the present invention, is intended to mean a collection of proteins obtained from or originating from a tumor cell or infectious agent. Included within the library of disease-derived proteins are general structural proteins and enzymes as well as disease-associated antigens.

Expression and display of the library of disease-derived proteins in lytic bacteriophage can be carried out using conventional cDNA or genomic phage display library construction methods with insertion of the cDNA or genomic library into commercially available lytic bacteriophage for expression and display on the surface of the phage. The cloned cDNA or gene can encode a complete protein or portions thereof. Methods for library construction are well-known in the art and can be found in general laboratory manuals such as Ausebel et al. (Eds) (1991) Current Protocols in Molecular Biology, New York; Greene Publishing & Wiley-Interscience; Sambrook, J. et al. (1989) Molecular Cloning: A Laboratory Manual (2nd ed.) Cold Spring Harbor: Cold Spring Harbor Laboratory Press.

Lytic bacteriophage are phage that lyse a host cell after the initial infection in order to release new phage particles. Lytic bacteriophage include lambda-phage, T3-phage, T4-phage, TB7-phage and T7-phage. Lytic bacteriophage vectors, such as lambda, T4 and T7 are of practical use since they are independent of E. coli secretion. Bacteriophage vectors are well-known in the art and commercially available. Examples of commercial T7 bacteriophage vectors include the T7SELECT series of vectors for engineering and packaging of DNA into T7 phage particles (NOVAGEN, Madison, Wis.). See also U.S. Pat. Nos. 5,223,409; 5,403,484; 5,571,698 and 5,766,905.

The library of phage can be used directly in the instant library screen, or alternatively amplified using an appropriate host (e.g., E. coli). The library of phage displaying the disease-derived proteins is subsequently assessed for the presence of disease-associated antigens by pulsing or contacting antigen presenting cells with the library of phage and detecting or measuring T cell responses during co-incubation of the antigen presenting cells and T helper cells. Advantageously, the antigen presenting cells naturally process and display disease-derived proteins on their surface so that those antigen presenting cells which present disease-associated antigens can be recognized by T helper cells. Examples of antigen presenting cells that can be used include, but are not limited to, antigen presenting cells such as EBV transformed B cell lines (Topalian, et al. (1994) Int. J. Cancer 58:69-79), monocytes and dendritic cells, and synthetic APC (see, e.g., U.S. Pat. No. 6,355,479).

Any conventional method can be employed to determine whether an antigen presenting cell is presenting an MHC class II-dependent disease-associated antigen which is recognized by a T helper cell. Such methods can be qualitative or quantitative to determine the degree of T helper cell recognition or stimulation. Exemplary methods include, but are not limited to, ⁵¹CR release cytotoxicity assays (Cerundolo, et al. (1990) Nature 345:449-452.); cytokine secretion assays such as γ-IFN, GM-CSF or TNF secretion (Schwartzentruber, et al. (1991) J. Immunology 146:3674-3681); or proliferation assays (e.g., a BrdU assay). A T helper cell which is stimulated (e.g., exhibits an increase in proliferation) in the presence of an APC is indicative of the presence of an MHC class II-dependent disease-associated antigen on the surface of said APC.

An MHC class II-dependent disease-associated antigen or epitope peptide thereof identified using the method of present invention finds application in the preparation of a vaccine for preventing or treating the disease associated with said antigen (i.e., cancer or infectious disease) as well as in the diagnosis of said disease or in the production of antibodies for treatment or diagnosis. Moreover, it is contemplated that the antigen presenting cells which presents the MHC class II-dependent disease-associated antigen can also be used in the preparation of a vaccine or in the production of antibodies.

For use in vaccines, diagnosis or antibody production, it is contemplated that the entire disease-associated antigen can be used or, alternatively, an immunogenic peptide or peptide epitope of said antigen can be used. An immunogenic peptide or peptide epitope is a peptide that contains an allele-specific motif or supermotif such that the peptide will bind an HLA molecule and induce a cellular or humoral immune response. Thus, immunogenic peptides of the invention are capable of binding to an appropriate HLA molecule and thereafter inducing a cytotoxic T lymphocyte (CTL) response, or a helper T lymphocyte (HTL) response, to the peptide.

An epitope is the collective features of a molecule, such as primary, secondary and tertiary peptide structure, and charge, that together form a site recognized by an immunoglobulin, T cell receptor or HLA molecule. Alternatively, an epitope can be defined as a set of amino acid residues which is involved in recognition by a particular immunoglobulin, or in the context of T cells, those residues necessary for recognition by T cell receptor proteins and/or Major Histocompatibility Complex (MHC) receptors. Epitopes can be isolated, purified or otherwise prepared/derived by humans. For example, epitopes can be prepared by isolation from a natural source, or they can be synthesized in accordance with standard protocols in the art. Synthetic epitopes can contain artificial amino acids, i.e., amino acid mimetics, such as D isomers of natural occurring L amino acids or non-natural amino acids such as cyclohexylalanine. Throughout this disclosure, the terms epitope and peptide are often used interchangeably.

Immunogenic peptides or peptide epitopes of the invention can be readily identified using conventional methods. For example, web-based algorithms can be used to analyze the amino acid sequence of a disease-associated antigen for potential human MHC class II binding epitopes. An exemplary algorithm is SYFPEITHI (Rammensee, et al. (1999) Immunogenetics 50:213) which ranks peptides according to a score taking into account the presence of primary and secondary MHC-binding anchor residues. Another exemplary algorithm is BIMAS (Parker, et al. (1994) J. Immunol. 152:163) which ranks potential binding according to the predicted half-time of dissociation of peptide/MHC complexes. Exemplary immunogenic peptides of RPL8 are disclosed in Table 3 and include SEQ ID NOs:5-249.

For use in accordance with the compositions and methods disclosed herein, a disease-associated antigen or immunogenic peptide thereof can be recombinantly-produced or chemically-synthesized using conventional methods well-known to the skilled artisan.

In general, recombinant production of a protein or peptide requires incorporation of nucleic acid sequences encoding said protein or peptide into a recombinant expression vector in a form suitable for expression of the protein or peptide in a host cell. A suitable form for expression provides that the recombinant expression vector includes one or more regulatory sequences operatively-linked to the nucleic acids encoding the protein or peptide in a manner which allows for transcription of the nucleic acids into mRNA and translation of the mRNA into the protein. Regulatory sequences can include promoters, enhancers and other expression control elements (e.g., polyadenylation signals). Such regulatory sequences are known to those skilled in the art and are described in Goeddel D. D., ed., Gene Expression Technology, Academic Press, San Diego, Calif. (1991). It should be understood that the design of the expression vector may depend on such factors as the choice of the host cell to be transfected and/or the level of expression required. Nucleic acid sequences or expression vectors harboring nucleic acid sequences encoding a disease-associated antigen or peptide can be introduced into a host cell, which may be of eukaryotic or prokaryotic origin, by standard techniques for transforming cells. Suitable methods for transforming host cells can be found in Sambrook, et al. (Molecular Cloning: A Laboratory Manual, 3rd Edition, Cold Spring Harbor Laboratory Press (2000)) and other laboratory manuals. The number of host cells transformed with a nucleic acid sequence will depend, at least in part, upon the type of recombinant expression vector used and the type of transformation technique used. Nucleic acids can be introduced into a host cell transiently, or more typically, for long-term expression the nucleic acid sequence is stably integrated into the genome of the host cell or remains as a stable episome in the host cell. Once produced, a disease-associated antigen or peptide can be recovered from culture medium as a secreted polypeptide, although it also may be recovered from host cell lysates when directly expressed without a secretory signal. When a disease-associated antigen or immunogenic peptide is expressed in a recombinant cell other than one of human origin, the disease-associated antigen or immunogenic peptide is substantially free of proteins or polypeptides of human origin. However, it may be necessary to purify the disease-associated antigen or peptide from recombinant cell proteins or polypeptides using conventional protein purification methods to obtain preparations that are substantially homogeneous as to the disease-associated antigen or immunogenic peptide.

In addition to recombinant production, a disease-associated antigen or immunogenic peptide may be produced by direct peptide synthesis using solid-phase techniques (Merrifield J. (1963) J. Am. Chem. Soc. 85:2149-2154).

Protein synthesis may be performed using manual techniques or by automation. Automated synthesis may be achieved, for example, using Applied Biosystems 431A Peptide Synthesizer (Perkin Elmer, Boston, Mass.). Various fragments of disease-associated antigen or immunogenic peptide can be chemically-synthesized separately and combined using chemical methods to produce a full-length molecule.

Whether recombinantly-produced or chemically-synthesized, a disease-associated antigen or immunogenic peptide can be further modified prior to use. For example, the peptides may be glycosylated, phosphorylated or fluorescently-tagged using well-known methods.

Disease-associated antigens or immunogenic peptides of the invention are useful for inducing an immune response to tumor cells or infectious agents. Accordingly, an MHC class II-dependent disease-associated T helper cell antigen of the present invention, or immunogenic peptide thereof, can be used as a vaccine either prophylactically or therapeutically. When provided prophylactically the vaccine is provided in advance of any evidence of disease. The prophylactic administration of the disease-associated antigen or immunogenic peptide vaccine should be administered as an effective amount to prevent or attenuate disease in a mammal. In one embodiment, mammals (e.g., humans, zoological animals, companion animals or livestock), at high risk for disease are prophylactically treated with the vaccines of this invention. Examples of such mammals include, but are not limited to, subjects with a family history of disease (e.g., genetically predisposed to cancer), subjects at risk of having a disease (e.g., individuals who have been exposed to cancer causing or infectious agents), subjects afflicted with a disease which has been treated and are therefore at risk for reoccurrence. When provided therapeutically, the vaccine is provided to enhance the subject's own immune response to the disease-associated antigen. The vaccine, which acts as an immunogen, can be a cell expressing the antigen or immunogic peptide (e.g., an APC as presented herein), cell lysate from cells transfected with a recombinant expression vector encoding the antigen or immunogic peptide, cell lysates from cells transfected with a recombinant expression vector encoding for the antigen or immunogic peptide, or a culture supernatant containing the expressed the antigen or immunogic peptide. Alternatively, the immunogen is a partially or substantially purified recombinant protein, peptide or analog thereof encoding for an antigen. The antigen or immunogic peptide can be conjugated with lipoprotein or administered in liposomal form or with adjuvant using conventional methodologies. As will be appreciated by the skilled artisan, a subject having, at risk of having, or suspected of having a disease will be administered a disease-associated antigen or immunogenic peptide for the disease being prevented or treated. By way of illustration, the instant RPL8 protein (SEQ ID NO:1) or immunogenic fragment or peptide thereof (e.g., SEQ ID NO:3 and SEQ ID NOs:5-249) is useful in the prevention or treatment of melanoma, glioma and ovarian cancer.

An effective amount of a disease-associated antigen or immunogenic peptide which can be used in accordance with the method of the invention is an amount which prevents, eliminates, alleviates, or reduces at least one sign or symptom of a cancer or infectious disease. For example, signs or symptoms associated with a cancer that can be monitored to determine the effectiveness of a tumor-associated antigen include, but are not limited to, tumor size and anti-tumor-associated antigen antibody production. Similarly, effectiveness of an infectious agent-associated antigen can be detected by monitoring antibody titer to the specific infectious agent-associated antigen. The amount of the disease-associated antigen or immunogenic peptide required to achieve the desired outcome of preventing, eliminating, alleviating or reducing a sign or symptom of disease will be dependent on the pharmaceutical composition employed, the patient and the condition of the patient, the mode of administration, and the type of disease being prevented or treated. Dose optimization is routine in the art and can be determined by the skilled clinician.

The disease-associated antigen or immunogenic peptide, which may be used alone or in combination, can be administered to a subject in need thereof, using any of the standard types of administration, such as intravenous, intradermal, subcutaneous, oral, rectal, and transdermal administration. Standard pharmaceutical carriers, adjuvants, such as saponins, GM-CSF, and interleukins and so forth can also be used. A generally recognized compendium of methods and ingredients of pharmaceutical compositions is Remington: The Science and Practice of Pharmacy, Alfonso R. Gennaro, editor, 20th ed. Lippincott Williams & Wilkins: Philadelphia, Pa., 2000. Further, proteins and peptides can be formulated into vaccines, as can dendritic cells, or other cells which present relevant MHC/peptide complexes. These proteins and peptides can also be used to form multimeric complexes of HLA/peptides, such as those described by Dunbar, et al. (1998) Curr. Biol. 8:413-416, wherein four peptide/MHC/biotin complexes are attached to a streptavidin or avidin molecule. Such complexes can be used to identify and/or to stimulate T cell precursors.

Similarly, the invention contemplates therapies wherein the nucleic acid molecule which encodes either full-length disease-associated antigen, or one or more of the relevant immunogenic peptides, in polytope form, is incorporated into a vector, such as an adenovirus-based vector, to render it transfectable into eukaryotic cells, such as human cells.

It is contemplated that a disease-associated antigen or immunogenic peptide can be conjugated to other species. The other species comprehended include all chemical species which can be fused to the protein or peptide without affecting the binding of the protein or peptide by T cells. Specific examples are, for example, other antigens such as epitopes which can elicit a separate immune response, carrier molecules which aid in absorption or protect the protein or peptide from enzyme action in order to improve the effective half-life.

As indicated, the invention involves, inter alia, an immune response to a disease-associated antigen or immunogenic peptide of interest. One ramification of this is the ability to monitor the course of a therapy. In this regard, a subject in need of the therapy receives a vaccination of a type described herein. Such a vaccination results, e.g., in a T cell response against cells presenting MHC/peptide complexes on their cells. The response also includes an antibody response, possibly a result of the release of antibody provoking proteins via the lysis of cells by the T cells. Hence, one can monitor the effect of a vaccine, by monitoring an immune response. As is indicated, supra, an increase in antibody titer or T cell count may be taken as an indicia of progress with a vaccine, and vice versa. The effects of a vaccine can also be measured by monitoring the T cell response of the subject receiving the vaccine. A number of assays can be used to measure the precursor frequency of these stimulated T cells. These include, but are not limited to, chromium release assays, TNF release assays, IFNγ release assays, an ELISPOT assay, and so forth. Changes in precursor T cell frequencies can be measured and correlated to the efficacy of the vaccine.

In addition to a disease-associated antigen or immunogenic peptide, a therapeutic of the invention also includes an antibody or antibodies reactive with a MHC class II-dependent disease-associated antigen or epitope peptide. In some embodiments, an antibody of the invention is raised against an antigen or epitope peptide identified by the instant screening method. In another embodiment, an antibody of the invention specifically binds an antigen or epitope peptide identified by the instant screening method. Such antibodies can be monoclonal and polyclonal and are made by conventional methods known to those skilled in the art. See, e.g., Current Protocols in Immunology, Wiley/Greene, NY; and Antibodies A Laboratory Manual Harlow, Harlow and Lane, Cold Spring Harbor Laboratory Press, 1989. Moreover, such antibodies can be natural or partially or wholly synthetically produced. All fragments or derivatives thereof which maintain the ability to specifically bind to a MHC class II-dependent disease-associated antigen are also included. The antibodies can be a member of any immunoglobulin class, including any of the human classes: IgG, IgM, IgA, IgD, and IgE.

Antibody fragments can be any derivative of an antibody which is less than full-length. In general, an antibody fragment retains at least a significant portion of the full-length antibody's specific binding ability. Examples of antibody fragments include, but are not limited to, Fab, Fab′, F(ab′)₂, scFv, Fv, diabody, or Fd fragments. The antibody fragment can be produced by any means. For instance, the antibody fragment can be enzymatically or chemically produced by fragmentation of an intact antibody or it can be recombinantly produced from a gene encoding the partial antibody sequence. The antibody fragment can optionally be a single-chain antibody fragment. Alternatively, the fragment can be multiple chains which are linked together, for instance, by disulfide linkages. The fragment can also optionally be a multi-molecular complex. A functional antibody fragment typically contains at least about 50 amino acids and more typically contains at least about 200 amino acids.

An antibody for use in the methods of the present invention can be generated using classical cloning and cell fusion techniques. For example, the antigen or epitope peptide of interest is typically administered (e.g., intraperitoneal injection) to wild-type or inbred mice (e.g., BALB/c) or transgenic mice which produce desired antibodies, or rats, rabbits or other animal species which can produce native or human antibodies. The antigen or epitope peptide can be administered alone, or mixed with adjuvant, or expressed from a vector (VEE replicon vector), or as DNA, or as a fusion protein to induce an immune response. Fusion proteins contain the antigen or epitope peptide against which an immune response is desired coupled to carrier proteins, such as histidine tag (his), mouse IgG2a Fc domain, β-galactosidase, glutathione S-transferase, keyhole limpet hemocyanin (KLH), or bovine serum albumin, to name a few. In these cases, the peptides serve as haptens with the carrier proteins. After the animal is boosted, for example, two or more times, the spleen is removed and splenocytes are extracted and fused with myeloma cells using the well-known processes (Kohler and Milstein (1975) Nature 256:495-497; Harlow and Lane (1988) Antibodies: A Laboratory Manual, Cold Spring Harbor Laboratory, New York). The resulting hybrid cells are then cloned in the conventional manner, e.g., using limiting dilution, and the resulting clones, which produce the desired monoclonal antibodies, are cultured.

Alternatively, antibodies which specifically bind a MHC class II-dependent disease-associated antigen or epitope peptide are produced by a phage display method. Methods of producing phage display antibodies are well-known in the art (e.g., Huse, et al. (1989) Science 246(4935):1275-81).

Selection of an antibody specific for a MHC class II-dependent disease-associated antigen or epitope peptide is based on binding affinity and can be determined by various well-known immunoassays including, enzyme-linked immunosorbent, immunodiffusion chemiluminescent, immunofluorescent, immunohistochemical, radioimmunoassay, agglutination, complement fixation, immunoelectrophoresis, and immunoprecipitation assays and the like which can be performed in vitro, in vivo or in situ. Such standard techniques are well-known to those of skill in the art (see, e.g., “Methods in Immunodiagnosis”, 2nd Edition, Rose and Bigazzi, eds. John Wiley & Sons, 1980; Campbell et al., “Methods and Immunology”, W.A. Benjamin, Inc., 1964; and Oellerich, M. (1984) J. Clin. Chem. Clin. Biochem. 22:895-904).

As with a MHC class II-dependent disease-associated antigen or epitope peptide, prevention or treatment with an antibody generally involves administering an effective amount of the antibody or antibody fragment to a subject in need of such treatment so that signs or symptoms associated with the disease are alleviated, prevented, or ameliorated. To produce an antibody which is more compatible with human vaccination, humanized chimeric antibodies may be desirable (see Morrison (1985) Science 229:1202; 01, et al. (1986) Biotechniques 4:214).

Antibodies of the invention are also useful in diagnostic, prognostic, or predictive methods to detect the presence of diseased tissues (e.g., tumors or infectious agents) via techniques such as ELISA, western blotting, or immunohistochemistry. The general method for detecting such an antigen provides contacting a sample with an antibody which specifically binds the antigen, so that an antibody-antigen complex is formed and detecting the antibody-antigen complex using any one of the immunoassays described above as well a number of well-known immunoassays used to detect and/or quantitate antigens (see, for example, Harlow and Lane (1988) supra). Such well-known immunoassays include antibody capture assays, antigen capture assays, and two-antibody sandwich assays.

Immunoassays typically rely on labeled antigens, antibodies, or secondary reagents for detection. These proteins may be labeled with radioactive compounds, enzymes, biotin, or fluorochromes. Of these, radioactive labeling can be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which can be employed in accordance with the present invention. The binding of these labels to antibodies or fragments thereof can be accomplished using standard techniques (e.g., Kennedy, et al. (1976) Clin. Chim. Acta 70:1-31; Schurs, et al. (1977) Clin. Chim Acta 81:1-40) and methods of detecting these labels are also well-known to the skilled artisan.

Antibodies disclosed herein can also be used for targeting therapeutic agents to cells expressing MHC class II-dependent disease-associated antigen. In this embodiment, therapeutic agents such as anti-neoplastic, anti-viral, anti-bacterial, or anti-fungal agents are operably linked to an antibody of the invention to facilitate targeting of the therapeutic agent to the target cell.

The invention is described in greater detail by the following non-limiting examples.

### Example 1

### Melanoma Patients

Melanoma patients #3472, 3507, 3522, 3523, and 3533 had metastatic lesions excised between 1 and 3 years ago. Patient 35 had a “low risk” primary melanoma of the superficial spreading type excised. The tumor was 0.69 mm in thickness and had a brisk lymphocytic infiltrate (Clark, et al. (1989) J. Natl. Cancer Inst. 81:1893-904). The primary lesion was excised approximately 23 years ago and there was no recurrence or metastasis since. PBMC were obtained from the patients' peripheral blood on the day of surgery (3522, 3523) or as late as 4 months after surgery (3507) with informed consent and under an approved protocol.

### Example 2

### Materials

Cell lines. Melanoma cell line WM35 was established from a primary melanoma (Satyamoorthy, et al. (1997) Melanoma Res. 7(Suppl 2):S35-42) and maintained in MCDB153-L15 medium (SIGMA-ALDRICH, St. Louis, Mo.) containing 2% fetal bovine serum (FBS). EBV-B35 was established from freshly isolated PBMC of patient 35 using 2.5 transforming U/cell of B95-8 virus according to known methods (Somasundaram, et al. (2003) supra). The cell line was maintained in RPMI 1640 medium with GLUTAMAX (GIBCO-INVITROGEN, Carlsbad, Calif.) supplemented with 10% FBS.

Th35-1A helper T cell clone was established by co-culturing PBMC with the autologous WM35 melanoma cell line, both derived from patient 35 (Somasundaram, et al. (2003) supra). COS-7L cells (GIBCO-INVITROGEN) were maintained in Dulbecco's Modification of Eagle's Medium (DMEM; GIBCO-INVITROGEN) supplemented with 10% FBS.

Antibodies. Anti-HLA class II antibody B33.1 is known in the art (Loza & Perussia (2001) Nature Immunology 2:917-924) and normal mouse IgG was obtained from Cappel-ICN (Costa Mesa, Calif.).

### Example 3

### cDNA Library Construction and Screening

EBV-B cells have been shown to present to T helper cells a tetanus toxoid cDNA fragment expressed by lysogenic filamentous phage (Somasundaram, et al. (2004) Clin. Exp. Immunol. 135:247-52). This approach was modified herein by using lytic bacteriophase (Rosenberg, et al. (1996) inNovations 6:1-6) to express a melanoma cDNA library. Messenger RNA was isolated from cultured WM35 cells using the FASTTRACK® 2.0 kit (INVITROGEN, Carlsbad, Calif.). Four μg of polyA⁺ RNA were converted to cDNA using the ORIENTEXPRESS system (EMD Biosciences NOVAGEN, San Diego, Calif.) and ligated into T7SELECT10-3b vector (EMD Biosciences NOVAGEN) according to the manufacturer's instructions. The ligated DNA was packed in vitro using T7 packing extract (library size was 3.2×10⁶ independent phage). The library was plate-amplified once in BLT5615 E. coli cells (EMD Biosciences NOVAGEN) and divided into 100 phage/pool. For screening, each pool was amplified once in liquid culture, and released phage were purified twice by PEG/NaCl precipitation. Phage titers were determined, and 3000 pfu were used to pulse EBV-B35 cells for co-culturing with Th35-1A cells in lymphocyte proliferation and interferon-γ release assays. Phage from one pool stimulated proliferation and interferon-γ release in Th35-1A cells.

### Example 4

### Assays

Lymphocyte Proliferation Assay. The lymphocyte proliferation assay was performed according to standard methods (Somasundaram, et al. (1995) J. Immunol. 155:3253-61). For screening of Th35-1A cell-reactivity with phage libraries, T helper cells (1-2×10⁴/well of 96-well round-bottom microtiter plates; CORNING, Corning, N.Y.) were cultured with irradiated autologous EBV-B cells (10⁴/well) pre-pulsed with 1-3×10³ phage. To determine T helper or PBMC reactivity with peptide, adherent monocytes (5×10⁴/well, obtained from PBMC) pre-pulsed with various concentrations (3.1-50 μM) of peptide were incubated with Th35-1A cells or PBMC (5×10⁴/well). T helper cells or PBMC were stimulated with peptide-pulsed monocytes once or twice. All incubations were at 37° C. for 5 days in RPMI 1640/GLUTAMAX medium supplemented with 10% heat-inactivated human AB serum (Gemini Bioproducts, West Sacramento, Calif.), 10 mM HEPES and 5×10⁻⁵ M 2-mercaptoethanol (both from SIGMA-ALDRICH). Proliferative responses of lymphocytes were determined using a standard [³H]-thymidine incorporation assay. All determinations were performed in triplicate. Results are expressed as counts per minute (cpm) incorporated into lymphocytes. The lymphocyte proliferation inhibition assay with anti-HLA class II antibody B33.1 was performed using established methods (Somasundaram, et al. (1995) supra).

IFN-γ Release Assay. Supernatants obtained 48 hours after T helper cell stimulation with phage-pulsed EBV-B cells were tested for the presence of IFN-γ using an ENDOGEN ELISA kit (Pierce Biotechnology, Inc., Rockford, Ill.).

### Example 5

### Peptide Design

DNA and deduced amino acid sequence comparisons were performed with the BLAST program provided by the National Center for Biotechnology Information. The amino acid sequence was deduced from the DNA sequence using EXPASY. DRB1*07011 binding epitopes were determined from the deduced amino acid sequence of the isolated cDNA clone by using the SYFPEITHI algorithm and Rammensee epitope prediction model (Rammensee, et al. (1999) Immunogenetics 50:213-9) and were limited to epitopes with a binding score>20. Selected peptides were synthesized and HPLC-purified. The following peptides were used: Val-Gly-Leu-Ile-Ala-Ala-Arg-Arg-Thr-Gly-Arg-Leu-Arg-Gly-Thr (SEQ ID NO:2), with a score of 24 (peptide #1, RPL8 position 235-249); Thr-Gly-Arg-Leu-Arg-Gly-Thr-Lys-Thr-Val-Gln-Glu-Lys-Glu-Asn (SEQ ID NO:3), with a score of 24 (peptide #2, RPL8 position 243-257); and Arg-Pro-Gly-Leu-Leu-Gly-Ala-Ser-Val-Leu-Gly-Leu-Asp-Asp-Ile (SEQ ID NO:350) with a score of 22 (control peptide, telomerase reverse transcriptase).

### Example 6

### Full-Length RPL8 Cloning

The GENERACER™ kit (INVITROGEN) and oligonucleotides based on the cDNA sequence of the phage that stimulated Th35-1A cell proliferation were used to determine the 5′ and 3′ end of RPL8 mRNA in WM35 cells. Both fragments (5′ and 3′ end) were sequenced and oligonucleotides were designed to clone full-length RPL8 cDNA by RT-PCR(SUPERSCRIPT™ III one-step RT-PCR with PLATINUM Taq; INVITROGEN).

### Example 7

### Northern Blot Analysis

Northern blot analysis of cells for the presence of RPL8 RNA was performed according to standard procedures. In short, total RNA was isolated from cultured cells using MICRO-TO-MIDI total RNA purification system (INVITROGEN). Ten μg of each RNA were separated on a 1.5% formaldehyde agarose gel and transferred onto a nylon membrane by electroblotting. The membrane was probed with [α-³²P]-dCTP-labeled, random-primed (REDIPRIME™ II random prime labeling system; Amersham Biosciences, Piscataway, N.J.), full-length RPL8 cDNA. RNA levels were compared using a STORM° PHOSPHORIMAGER system (GE Healthcare, Piscataway, N.J.). Assumption of equal loading was based on OD reading and ethidium bromide staining signal of ribosomal RNA. There was no correlation between RNA levels and recognition of cell lysates by Th35-1A cells.

### Example 8

### Statistical Analyses

Differences between experimental and control values were analyzed for significance by Student's 2-sided t-test.

What is claimed is:

1. A method for identifying a MHC class II-dependent disease-associated T helper cell antigen comprising expressing a library of disease-derived proteins in lytic bacteriophage; presenting antigens of the library of disease-derived proteins on the surface of MHC class II-positive antigen presenting cells (APC); contacting the APC with T helper cells and determining T helper cell recognition, wherein the recognition by a T helper cells is indicative of said APC presenting a MHC class II-dependent disease-associated T helper cell antigen.

2. A MHC class II-dependent disease-associated T helper cell antigen identified by the method of claim 1.

3. A vaccine comprising the MHC class II-dependent disease-associated T helper cell antigen identified by the method of claim 1.

4. The vaccine of claim 3, wherein the MHC class II-dependent disease-associated T helper cell antigen is a tumor-associated T helper cell antigen.

5. The vaccine of claim 3, wherein the MHC class II-dependent disease-associated T helper cell antigen is an infectious agent-associated T helper cell antigen.

6. A method for inducing an immune response to a MHC class II-dependent disease-associated T helper cell antigen comprising contacting a T helper cell with the MHC class II-dependent disease-associated T helper cell antigen of claim 2 so that an immune response is induced.

7. A method for preventing or treating cancer comprising administering the vaccine of claim 4 to a subject in need thereof so that cancer in the subject is prevented or treated.

8. A method for preventing or treating an infectious disease comprising administering the vaccine of claim 5 to a subject in need thereof so that infectious disease in the subject is prevented or treated.

9. An isolated antibody which specifically binds the MHC class II-dependent disease-associated T helper cell antigen of claim 2 or an epitope peptide thereof.

10. The isolated antibody of claim 9, wherein the MHC class II-dependent disease-associated T helper cell antigen is a tumor-associated T helper cell antigen.

11. The isolated antibody of claim 9, wherein the MHC class II-dependent disease-associated T helper cell antigen is an infectious agent-associated T helper cell antigen.

12. A method for preventing or treating cancer comprising administering the antibody of claim 10 to a subject in need thereof so that cancer in the subject is prevented or treated.

13. A method for preventing or treating an infectious disease comprising administering the antibody of claim 11 to a subject in need thereof so that infectious disease in the subject is prevented or treated.
